(19) United States
(12) Patent Application Publication
(10) Pub. No.: US 2026/0098288 A1
(43) Pub. Date: Apr. 9, 2026

(54) MOLECULAR INTERACTION DETECTION AND PROFILING

(71) Applicant: NAVINCI DIAGNOSTICS AB, Uppsala (SE)

(72) Inventors: Bo XU, Uppsala (SE); Ulf LANDEGREN, Uppsala (SE); Axel KLAESSON, Uppsala (SE); Agata Zieba WICHER, Uppsala (SE)

(73) Assignee: NAVINCI DIAGNOSTICS AB, Uppsala (SE)

(21) Appl. No.: 19/112,710

(22) PCT Filed: Sep. 22, 2023
(86) PCT No.: PCT/EP2023/076250

§ 371 Date: Mar. 18, 2025

(30) Foreign Application Priority Data
Sep. 23, 2022 (GB) 2213956.2

(51) Int. Cl. C12Q 1/6804; C12Q 1/6818; G01N 33/68

(52) U.S. Cl. CPC C12Q 1/6804; C12Q 1/6818; G01N 33/6845; G01N 2458/10

(57) ABSTRACT

The present invention provides a method for detecting two target molecules in a sample, both as individual molecules and in proximity, or interaction with one another. The method involves performing three separate assay reactions to detect the first and second target molecules, and their interaction, using proximity probes which are common between the three assay reactions, and nucleic acid reagents which interact with the probes. In particular the methods use unique nucleic acid substrate molecules, such as padlock probes, to detect probes bound to their target and to determine when probes for the two targets are in close proximity, indicating an interaction between the targets.

### FIELD

The present invention relates generally to a method for detecting two proximal or interacting target molecules in a sample. The method allows the detection of the two target molecules individually, and also the detection of proximity between the molecules, which may be indicative of an interaction between them, and determination of the amounts of each molecule which are proximal or interacting with each other. The method uses probes including proximity probes to bind the target molecules, and nucleic acid reagents which interact with the probes, in particular unique nucleic acid substrate molecules (for example padlock probes), to detect probes bound to their target and to determine when probes for the two targets are in close proximity, indicating an interaction between the targets.

### BACKGROUND

Many methods have been developed in the art to detect target molecules such as proteins and nucleic acids. Generally, it is convenient to be able to detect proteins in multiplex, but current multiplexed protein detection methods on the market focus on detecting the expression levels of individual proteins, and functional aspects of those proteins, such as their protein-protein interactions (PPIs) or posttranslational modifications (PTMs), are neglected in those methods.

Methods have been developed to study protein interactions, including notably proximity assays, which rely on an interaction between a pair of proximity probes which, when both probes of the pair have become bound to their respective targets in proximity (which occurs when the respective targets have interacted), are able to generate a signal indicative of the interaction; no signal is generated when the targets are not in proximity (have not interacted). Such proximity assays are typified by the in situ proximity ligation assay (isPLA), which is a powerful and widely used tool for detecting PPIs or PTMs. The limitation of the assay is that it only detects the proximity event (i.e. the interaction), while the expression levels of proteins undergoing PPI or PTM cannot be detected. Besides this, in situ PLA has so far only rarely been applied in a multiplexing context.

Proximity probes used in proximity assays typically comprise a binding domain capable of binding directly (primary binding partner) or indirectly (secondary binding partner) to a target analyte, and a nucleic acid domain. The nucleic acid domains of a pair of proximity probes interact directly with each other, e.g. by hybridization and extension, and/or by ligation, or indirectly via another nucleic acid molecule, to generate a nucleic acid product that may be detected in order to detect the target of the proximity assay (which as noted above may be a complex of interacting proteins, but need not necessarily be so; proximity assays are also used to detect individual target molecules, where both proximity probes of the proximity probe pair bind to the same target molecule). A ligation product of a proximity assay may be generated by ligation of the nucleic acid domains to each other, or to another molecule, or by ligation of one or more other nucleic acid molecules which bind to the nucleic acid domains. Thus, one or more nucleic acid molecules may bind to both of the nucleic acid domains of the two proximity probes when they are both bound in proximity to their respective targets, and the ligation of the nucleic acid molecule(s) may be templated by one or both of the nucleic acid domains of the proximity probes. The nucleic acid molecule(s) which are ligated in this way may be a padlock probe, which is circularized by the ligation. Such a configuration represents the classical format of an in situ PLA.

A padlock probe is a circularisable probe comprising one or more linear oligonucleotides which may be ligated together to form a circle. Padlock probes are well known and widely used in the art. A padlock probe is typically a linear oligoncucleotide with sequences at its 3′ and 5′ ends which are complementary to sequences in a target nucleic acid molecule. The target-complementary binding sequences are connected by an intervening “backbone” region which does not bind to the target molecule. Hybridisation of the ends of the probe to the target nucleic acid molecule brings the ends of the probe into a position where they may be ligated to each, either directly, where they have hybridised adjacent to one another, or indirectly, where there is a gap between the hybridised ends. The gap may be filed by a gap oligonucleotide (which can be seen as a part of a 2-part padlock probe) or by extension of the hybridised 3′ end of the padlock probe to generate a ligatable 3′ end adjacent the hybridised 5′ end of the padlock probe. The resulting circular nucleic acid molecule can be detected by sequencing or qPCR etc, or by rolling circle amplification (RCA) using the circularised padlock as the RCA template. The RCA product can then be detected, e.g. by using probes to detect a target sequence, or by digestion of the RCA product into monomers followed by sequencing.

RCA utilises a strand-displacing polymerase to extend a primer which is hybridised to a circular template (the strand displacing activity displaces the primer and effectively causes the circle to “roll”). The addition of a polymerase and nucleotides starts the synthesis reaction, i.e. polymerisation. As the rolling circle template is endless, the resultant RCA product is a long single stranded nucleic acid molecule composed of catenated tandem repeats that are complementary to the rolling circle template (i.e. a concatemer). Such a concatemer typically forms a ball, or “blob”, which may readily be visualised and counted. For this reason RCA has been widely adopted as an amplification and/or detection technique in various nucleic acid-based assays, commonly in conjunction with padlock probes.

Other methods for detection of interactions between two analytes (e.g. protein-protein interactions) include the MolBoolean method described in WO 2018/147794. The MolBoolean method utilises a single-stranded substrate nucleic acid which, upon interaction with the nucleic acid domain of a proximity probe forms a double-stranded segment comprising a cleavage site into which a tag is inserted corresponding to an analyte of interest. Insertion of a single tag into the substrate nucleic acid identifies the presence of a non-interacting analyte, while insertion of both tags (corresponding to both analytes) into the substrate nucleic acid identifies an interaction between the analytes. In this way the amount of each analyte interacting with the other can be identified. The present method is advantageous over the MolBoolean method in that no enzymatic cleavage step is required, and further by generating distinct nucleic acid products, for example by using different padlock probes or other nucleic acid-based reagents that interact with the nucleic acid domains of proximity probes, the method can be easily multiplexed.

### SUMMARY

The new method herein has been developed to overcome limitations of the methods discussed above, to enable detection in the same assay of both an interaction, or more particularly proximity, which may be indicative of an interaction, between two target molecules, and the individual expression levels of the two participating molecules in a manner which may readily be multiplexed. The method uses proximity probes which bind to the two respective target molecules, in a manner which enables 3 separate detection reactions to be performed, to generate three distinct signals (e.g. detectable reaction products) which may be detected separately to detect the two individual proteins and the interaction. A proximity probe pair is used to detect the target interaction, and the individual members of the proximity probe pair are also used separately in the detection of the individual target molecules. Conveniently, different padlock probes may be used in conjunction with the proximity probes, capable of interacting with one, or with both, of the nucleic acid domains of the proximity probes, to generate 3 distinct circularised RCA templates which may be detected by RCA to detect the interaction and the individual members of that interaction. However, more generally any nucleic acid reagents capable of hybridising to the nucleic acid domains of the proximity probes and giving rise to detectable signals or products may be used, and different reagents may be used in the 3 separate detection reactions. Thus, for example, a padlock probe capable of hybridising to both nucleic acid domains of the proximity probe pair may be used to detect the interaction, and separate detection probes, each capable respectively of binding specifically to one of the nucleic acid domains of the proximity probe pair but not to the other, may be used to detect each of the two proximity probes separately, and hence the two individual target molecules to which the proximity probes have become bound. Other nucleic acid reagents include other substrates for generation of a nucleic acid product, such as HCR reagents, and reagents for the build-up of a nucleic acid hybridisation-based signal amplification system, such as a branched DNA amplification system.

Accordingly, in a broad first aspect, provided herein is a method for detecting two target molecules in a sample, and detecting an interaction between the two target molecules, the method comprising:
(i) contacting the sample with (a) a first probe, or a first proximity probe pair, for detection of a first target molecule and (b) a second probe, or a second proximity probe pair for detection of a second target molecule;
   wherein said probes each comprise a binding domain capable of binding directly or indirectly to their target molecule and a nucleic acid domain, and said first and second probes, or one of the probes of the first proximity probe pair and one of the probes of the second proximity probe pair, together form a third proximity probe pair for detection of the interaction between the first and second target molecules;
(ii) performing a first assay reaction to detect the nucleic acid domain of the first probe or of at least one member of the first proximity probe pair, thereby detecting the first target molecule;
(iii) performing a second assay reaction to detect the nucleic acid domain of the second probe or of at least one member of the second proximity probe pair, thereby detecting the second target molecule;
(iv) performing a third assay reaction to detect an interaction between the first and second target molecules, wherein said third assay reaction is a proximity assay using the third proximity probe pair, wherein, when said first and second target molecules are present in proximity in an interaction, the nucleic acid domains of the third proximity probe pair interact with each other directly or indirectly to generate a nucleic acid product, and said product is detected to detect the interaction between the first and second target molecules.

In an embodiment the first and second assays comprise quantifying the amount of target molecule which is detected, and the third assay comprises quantifying the amount of target molecules which are present in the interaction.

The first, second and third assay reactions are separate independent reactions which are carried out as part of the same assay method. They may be performed simultaneously or sequentially. Each assay reaction is separately performed. More particularly, each assay reaction produces a separate signal. In an embodiment, each assay reaction results in, or produces, a separate product which is detected. Thus, the method as a whole produces three separate reaction products, and each product separately is indicative respectively of the two individual target molecules and the target molecule interaction.

In an embodiment of the method, nucleic acid reagents are used which hybridise to the nucleic acid domains of the proximity probes, which reagents allow the detection of that probe or of a proximity probe pair which have bound together in proximity. The nucleic acid reagents may give rise to a detectable signal, for example, they may be detectably labelled or they result in the generation of a product, e.g. a hybridisation, extension, ligation or amplification product, which may be detected, in order to detect, respectively, the two individual target molecules and the interaction.

The method may employ at least two nucleic acid reagents. In an embodiment, one nucleic acid reagent may participate in two of the assay reactions. In another embodiment three nucleic acid reagents may be used, a separate reagent for each assay reaction.

In a particular embodiment:
(a) the first assay reaction utilises a first nucleic acid reagent capable of hybridising to the nucleic acid domain of the first probe or of at least one member of the first proximity probe pair, and said first reagent or the hybridisation thereof is detected;
(b) the second assay reaction utilises a second nucleic acid reagent capable of hybridising to the nucleic acid domain of the second probe or of at least one member of the second proximity probe pair, and said second reagent or the hybridisation thereof is detected; and
(c) the third assay either (i) utilises a third nucleic acid reagent capable of hybridising to both the nucleic acid domains of the third proximity probe pair to generate the nucleic acid product of the proximity probe interaction, or (ii) utilises one of the first or second nucleic acid reagents, which is capable of hybridising also to the nucleic acid domain of the other member of the third proximity probe pair to generate the nucleic acid product of the third proximity probe pair interaction.

In an embodiment, the nucleic acid domains of the proximity probes comprise tag sequences which are detected.

In an embodiment, the nucleic acid domain of said first probe or of one member of the first proximity probe pair comprises a first tag sequence which is detected to detect the first target molecule; the nucleic acid domain of the second probe or of one member of the second proximity probe pair comprises a second tag sequence which is detected to detect the second target molecule; and the nucleic acid domains of said first and second probes or the members of the first and second proximity pairs which constitute the third proximity probe pair each additionally comprise a separate region of complementarity capable of mediating the interaction between the nucleic acid domains of the third proximity probe pair. In a particular embodiment this region of complementarity may be a binding site, or hybridisation site, for a nucleic acid reagent capable of hybridising to both the nucleic acid domains of the third proximity pair.

In an embodiment, the first and second tag sequences constitute binding/hybridisation sites for the first and second nucleic acid reagents respectively. In such an embodiment, the separate regions of complementarity in the nucleic acid domains of the third proximity probe pair comprise or constitute binding/hybridisation sites for a third nucleic acid reagent.

In an embodiment, the first and second assay reactions generate first and second nucleic acid products from or using the first and second nucleic acid reagents respectively, and a third nucleic acid product is generated in the third assay reaction.

In an embodiment, first, second and third nucleic acid products are generated in the first, second and third assay reactions respectively. In a more particular embodiment, the first, second, and third nucleic acid products are generated from or using first, second and thirds reagents respectively. The three nucleic acid products are distinguishable from one another by sequence and are detected to detect the first and second target molecules and the interaction between them.

In an embodiment, the nucleic acid products are amplified and the resulting amplicons are detected.

The nucleic acid reagents may be substrates for generating nucleic acid products. In an embodiment, the substrate is a padlock probe. In this embodiment, the product is a circularised nucleic acid molecule, which may be amplified by RCA to produce a RCA product (RCP). In another embodiment the substrate is a substrate for a HCR reaction or for a branched DNA amplification reaction.

Accordingly, in a more particular version of the first aspect, provided herein is a method for detecting two target molecules in a sample, and detecting an interaction between the two target molecules, the method comprising:
(i) contacting the sample with (a) a first probe, or a first proximity probe pair, for detection of a first target molecule and (b) a second probe, or a second proximity probe pair for detection of a second target molecule;
wherein said probes each comprise a binding domain capable of binding directly or indirectly to the target molecule and a nucleic acid domain, and said first and second probes, or one of the probes of the first proximity probe pair and one of the probes of the second proximity probe pair, together form a third proximity probe pair for detection of the interaction between the first and second target molecules;
(ii) contacting the probes with at least first and second nucleic acid substrate molecules, wherein the first substrate molecule hybridises with the nucleic acid domain of the first probe or one of the probes of the first proximity probe pair and the second substrate molecule hybridises with the nucleic acid domain of the second probe or one of the probes of the second proximity probe pair;
(iii) performing a first assay reaction to detect a first target molecule using the first probe or at least one member of the first proximity probe pair, and generating a first nucleic acid product from the first nucleic acid substrate;
(iv) performing a second assay reaction to detect a second target molecule using the second probe or at least one member of the second proximity probe pair, and generating a second nucleic acid product from the second nucleic acid substrate;
(v) performing a third assay reaction to detect an interaction between the first and second target molecules using the third proximity probe pair, and when said first and second target molecules are present in proximity in an interaction, generating a third nucleic acid product;
wherein the first, second and third nucleic acid products are distinguishable from one another by sequence;
(vi) optionally generating amplification products of or from said first, second, and where present, third nucleic acid products; and
(vii) detecting said nucleic acid products or amplification products, wherein the first nucleic acid or amplification product indicates the first target molecule, the second nucleic acid or amplification product indicates the second target molecule, and the third nucleic acid or amplification product indicates the interaction between the first and second target molecules.

In an embodiment, step (ii) above further comprises contacting the probes with a third substrate molecule, which hybridises to both nucleic acid domains of the third proximity probe pair, and step (v) comprises generating the third nucleic acid product from said third substrate.

In an alternative embodiment, the third nucleic acid product is generated from interaction of the first or second substrate molecule with the nucleic acid domains of the third proximity probe pair.

In an embodiment, the nucleic acid substrates are padlock probes, which may be provided in one or more parts (e.g. two parts). The padlock probes may be ligated, directly or indirectly, using the nucleic acid domains to which they hybridise as ligation templates. The ligated circularised products may be amplified by RCA.

In all embodiments which involve contacting the proximity probes with a nucleic acid reagent (e.g. substrate), the contacting may be before, during or after contacting of the sample with the proximity probes. This may depend on the precise method format and method steps. Thus, in some embodiments the nucleic acid reagent, particularly where it is a substrate such a padlock probe, may be pre-hybridised to the nucleic acid domain of a proximity probe before the proximity probe is contacted with the sample. In other embodiments, the nucleic acid reagent may be contacted with the proximity probe after it has been contacted with the sample, more particularly after it has bound to its target molecule, e.g. where the detection reagent is a detection probe for detection of the first or second proximity probe.

In a particular embodiment, the method may be defined as a method for detecting two target molecules in a sample, and detecting an interaction between the two target molecules, the method comprising:
(i) contacting the sample with:
   (a) a first proximity probe, for the first target molecule, wherein the first proximity probe comprises a binding domain capable of binding directly or indirectly to the first target molecule and a first nucleic acid domain comprising one or more (e.g. two) single-stranded regions, wherein the first nucleic acid domain comprises a first padlock binding site for a first padlock probe, and a hybridisation sequence capable of hybridising to a third padlock probe, the first padlock binding site and the hybridisation sequence both being located within the one or more single-stranded regions;
   (b) a second proximity probe, for the second target molecule, wherein the second proximity probe comprises a binding domain capable of binding directly or indirectly to the second target molecule and a second nucleic acid domain comprising one or more single-stranded regions, wherein the second nucleic acid domain comprises a second padlock binding site for a second padlock probe and a hybridisation sequence capable of hybridising to the third padlock probe, the second padlock binding site and the hybridisation sequence both being located within the one or more single-stranded regions;
   (c) a first padlock probe which comprises at its 5′ and 3′ ends target binding sequences capable of hybridising to the first padlock binding site of the first proximity probe;
   (d) a second padlock probe which comprises at its 5′ and 3′ ends target binding sequences capable of hybridising to the second padlock binding site of the second proximity probe; and
   (e) a third padlock probe, which comprises:
      (I) a single circularisable oligonucleotide comprising target binding sequences at its 5′ and 3′ ends capable of hybridising to the hybridisation sequence of the second proximity probe, and in the backbone region between the 5′ and 3′ ends an anchor sequence capable of hybridising to the hybridisation sequence of the first proximity probe, and wherein when applied to the sample the anchor sequence is hybridised to its complementary hybridisation sequence of the first proximity probe; or
      (II) two circularisation oligonucleotides together forming a two-part padlock probe, each circularisation oligonucleotide comprising at its 5′ and 3′ ends a first target binding sequence capable of hybridising to the hybridisation sequence of the first proximity probe and a second target binding sequence capable of hybridising to the hybridisation sequence of the second proximity probe, such that when the first and second proximity probes are in proximity (i.e. when the first and second target molecules are interacting), each circularisation oligonucleotide can hybridise to the nucleic acid domains of both proximity probes, and the respective 5′ and 3′ ends of the two circularisation oligonucleotides are brought into juxtaposition for ligation, directly or indirectly, to each other to form a circle;
(ii) where the 5′ and 3′ ends of the padlock probes have hybridised to their respective binding sites or hybridisation sequences with a gap between them, performing a gap-filling reaction (by hybridising a gap oligonucleotide and/or by extension of the 3′ end), and ligating the hybridised padlock probes, thereby generating a first circular nucleic acid product from the first padlock probe, a second circular nucleic acid product from the second padlock probe, and a third circular nucleic acid product from the third padlock probe, wherein the first, second and third circular nucleic acid products are distinguishable from one another by sequence (e.g. they may each comprise a distinguishable tag sequence, or a unique identifier sequence);
(iii) optionally amplifying the first, second and third circular nucleic acid products by rolling circle amplification (RCA) to generate a first, second and third RCA product (RCP); and
(iv) detecting the first, second and third circular nucleic acid product or RCP wherein the first circular nucleic acid product or RCP indicates the first target molecule, the second circular nucleic acid product or RCP indicates the second target molecule, and the third circular nucleic acid product or RCP indicates the interaction between the two target molecules.

It will be seen that the hybridisation sequences in the first and second nucleic acid domains may be target binding sites for the target binding regions of the third padlock probe or they may be an anchor binding sequence for an anchor sequence in the backbone region of the third padlock probe. Thus, depending on the probe configuration, the hybridisation sequence may be a target binding sequence for the third padlock probe, i.e. it may provide a ligation template for the third padlock probe, or it may be a capture sequence for the third padlock probe, i.e. by means of which the padlock probe may be attached to the nucleic acid domain of a proximity probe.

The first and second proximity probes of the above embodiment may be replaced by proximity probe pairs specific for each of the two target molecules.

Accordingly, another particular embodiment of the first aspect is a method for detecting two target molecules in a sample, and detecting an interaction between the two target molecules, the method comprising:
(i) contacting the sample with:
   (a) a first proximity probe pair comprising a first and second proximity probe for the first target molecule, each proximity probe comprising a binding domain capable of binding directly or indirectly to the first target molecule and a nucleic acid domain comprising one or more (e.g. two) single-stranded regions, wherein the nucleic acid domain of the first proximity probe comprises a first padlock binding site for a first padlock probe and the nucleic acid domain of the second proximity probe comprises a hybridisation sequence capable of hybridising to a third padlock probe, the first padlock binding site and the hybridisation sequence both being located within the one or more single-stranded regions;
   (b) a second proximity probe pair comprising a first and second proximity probe for the second target molecule, each proximity probe comprising a binding domain capable of binding directly or indirectly to the second target molecule and a nucleic acid domain comprising one or more single-stranded regions, wherein the nucleic acid domain of the first proximity probe comprises a second padlock binding site for a second padlock probe and the nucleic acid domain of the second proximity probe comprises a hybridisation sequence capable of hybridising to the third padlock probe, the second padlock binding site and the hybridisation sequence being located within the one or more single-stranded regions;
   (c) a first padlock probe which comprises at its 5′ and 3′ ends target-binding sequences capable of hybridising to the first padlock binding site of the first proximity probe of the first proximity probe pair;
   (d) a second padlock probe which comprises at its 5′ and 3′ ends target-binding sequences capable of hybridising to the second padlock binding site of the first proximity probe of the second proximity probe pair;
   (e) a third padlock probe, which comprises:
      (I) a single circularisable oligonucleotide comprising target-binding sequences at its 5′ and 3′ ends capable of hybridising to the hybridisation sequence of the second proximity probe of the second proximity probe pair, and in the backbone region between the 5′ and 3′ ends an anchor sequence capable of hybridising to the hybridisation sequence of the second proximity probe of the first proximity probe pair, and wherein when applied to the sample the anchor sequence is hybridised to its complementary hybridisation sequence of the second proximity probe of the first proximity probe pair; or
      (II) two circularisation oligonucleotides together forming a two-part padlock probe, each circularisation oligonucleotide comprising at its 5′ and 3′ ends a first target-binding sequence capable of hybridising to the hybridisation sequence of the second proximity probe of the first proximity probe pair and a second target-binding sequence capable of hybridising to the hybridisation sequence of the second proximity probe of the second proximity probe pair, such that when the first and second proximity probes are in proximity (i.e. when the first and second target molecules are interacting), each circularisation oligonucleotide can hybridise to the nucleic acid domains of both second proximity probes, and the respective 5′ and 3′ ends of the two circularisation oligonucleotides are brought into juxtaposition for ligation, directly or indirectly, to each other to form a circle;
(ii) where the 5′ and 3′ ends of the padlock probes have hybridised to their respective binding sites or hybridisation sequences with a gap between them, performing a gap-filling reaction (by hybridising a gap oligonucleotide and/or by extension of the 3′ end), and ligating the hybridised padlock probes, thereby generating a first circular nucleic acid product from the first padlock probe, a second circular nucleic acid product from the second padlock probe, and a third circular nucleic acid product from the third padlock probe, wherein the first, second and third circular nucleic acid products are distinguishable from one another by sequence (e.g. they may each comprise a distinguishable tag sequence, or a unique identifier sequence);
(iii) amplifying the first, second and third circular nucleic acid products by rolling circle amplification (RCA) to generate a first, second and third RCA product (RCP); and
   (v) detecting the first, second and third RCP, wherein the first RCP indicates the first target molecule, the second RCP indicates the second target molecule, and the third RCP indicates the interaction between the two target molecules.

In a more particular embodiment, the nucleic acid domain of the second proximity probe of the first proximity probe pair additionally comprises a hybridisation sequence capable of hybridising to a complementary anchor sequence in the first padlock probe, and the nucleic acid domain of the second proximity probe of the second proximity probe pair additionally comprises a hybridisation sequence capable of hybridising to a complementary anchor sequence in the second padlock probe. In this way a proximity event is also required to detect the individual first and second target molecules.

In a still further particular embodiment, there is provided a method for detecting two target molecules in a sample, and detecting an interaction between the two target molecules, the method comprising:
(i) contacting the sample with:
   (a) a first proximity probe for the first target molecule, wherein the first proximity probe comprises a nucleic acid domain (e.g. a single-stranded nucleic acid domain) hybridised to a first padlock probe (more particularly to the backbone region thereof), wherein the 5′ and 3′ ends of the first padlock probe are hybridised to a blocking oligonucleotide;
   (b) a second proximity probe for the second target molecule, wherein the second proximity probe comprises a nucleic acid domain comprising one or more single-stranded regions, and wherein the nucleic acid domain comprises a first padlock binding site capable of hybridising to the 5′ and 3′ ends of the first padlock probe, and a second padlock binding site for a second padlock probe, the padlock binding sites both being located within the one or more single-stranded regions; and
   (c) a second padlock probe, which comprises at its 5′ and 3′ ends target-binding sequences capable of hybridising to the second padlock binding site of the second proximity probe;
such that when the first and second proximity probe are in proximity (i.e. when the first and second target molecules have interacted) the blocking oligonucleotide is displaced from the first padlock probe by the single stranded region comprising the first padlock binding site of the second proximity probe, and when the first and second proximity probes are not in proximity the first padlock probe remains bound to the blocking oligonucleotide (and does not bind to the nucleic acid domain of the second proximity probe),
wherein the blocking oligonucleotide and/or the first padlock binding site of the second proximity probe comprise a gap sequence located between complementary binding sites capable of hybridising to the 5′ and 3′ ends of the first padlock probe, such that the hybridised 3′ and 5′ ends of the first padlock probe are separated by a gap (and cannot be ligated to one another);
(ii) when the 5′ and 3′ ends of the padlock probes have hybridised to their respective binding sites with a gap between them, performing a gap-filling reaction (by hybridising a gap oligonucleotide and/or by extension of the 3′ end), and ligating the hybridised padlock probes, thereby generating a first circular nucleic acid product from the first padlock probe hybridised to the blocking oligonucleotide, a second circular nucleic acid product from the second padlock probe, and a third circular nucleic acid product from the first padlock probe hybridised to the first padlock binding site of the second proximity probe, wherein the first, second and third circular nucleic acid products are distinguishable from one another by sequence (e.g. they may each comprise a distinguishable tag sequence, or a unique identifier sequence);
(iii) amplifying the first, second and third circular nucleic acid products by rolling circle amplification (RCA) to generate a first, second and third RCA product (RCP); and
(iv) detecting the first, second and third RCP, wherein the first RCP indicates the first target molecule not interacting with the second target molecule, the second RCP indicates the second target molecule, and the third RCP indicates the interaction between the two target molecules.

In a more specific embodiment of this version of the method, the blocking oligonucleotide comprises complementary binding sites for the 5′ and 3′ ends of the first padlock probe separated by an intervening gap sequence, such that the hybridised 3′ and 5′ ends are separated by a gap.

Another particular embodiment of the first aspect is a method for detecting two target molecules in a sample, and detecting an interaction between the two target molecules, the method comprising:
(i) contacting the sample with:
   (a) a first proximity probe pair comprising a first and second proximity probe for the first target molecule, wherein the first proximity probe comprises nucleic acid domain (e.g. a single-stranded nucleic acid domain) comprising a first padlock binding site, and the second proximity probe comprises nucleic acid domain (e.g. a single-stranded nucleic acid domain) hybridised to a first padlock probe (more particularly to the backbone region thereof) which comprises at its 5′and 3′ends binding sequences capable of hybridising to the first padlock binding site (i.e. of the first proximity probe of the first pair); and
   (b) a second proximity probe pair comprising a first and second proximity probe for the second target molecule, wherein the first proximity probe comprises a nucleic acid domain (e.g. a single-stranded nucleic acid domain) comprising a second padlock binding site, and the second proximity probe comprises a nucleic acid domain (e.g. a single-stranded nucleic acid domain) hybridised to a second padlock probe (more particularly to the backbone region thereof) which comprises at its 5′ and 3′ ends binding sequences capable of hybridising to the second padlock binding site (i.e. of the first proximity probe of the second pair);
wherein the binding sequences of the first padlock probe are also capable of hybridising to the second padlock binding site, and/or the binding sequences of the second padlock probe are also capable of hybridising to the first padlock binding site;
wherein the first and second padlock probes each comprise an identifier sequence, and the first and/or second padlock binding sites comprise a gap sequence (which may be or comprise e.g. a tag or barcode sequence) located between complementary binding sites capable of hybridising to the 5′ and 3′ ends of the respective padlock probes;
(ii) where the 5′ and 3′ ends of a padlock probe have hybridised to their respective binding sites with a gap between them, performing a gap-filling reaction (by hybridising a gap oligonucleotide and/or by extension of the 3′ end), and ligating the hybridised padlock probes, thereby generating a first circular nucleic acid product from the first padlock probe hybridised to the first padlock binding site, a second circular nucleic acid product from the second padlock probe hybridised to the second padlock binding site, and a third and, optionally, fourth circular nucleic acid product from the first padlock probe hybridised to the second padlock binding site and/or the second padlock probe hybridised to the first padlock binding site, wherein the first, second, third and optionally fourth circular nucleic acid products are distinguishable from one another by sequence (e.g. they may each comprise each comprise a unique combination of identifier sequence and optional gap-fill sequence;
(iii) amplifying the first, second, third and optional fourth circular nucleic acid products by rolling circle amplification (RCA) to generate a first, second, third and, optionally, fourth, RCA product (RCP); and
(iv) detecting the first, second, third and optional fourth RCP (e.g. by detecting their unique combination of identifier and optional gap-fill sequences), wherein the first RCP indicates the first target molecule, the second RCP indicates the second target molecule, and the third RCP, and optional fourth RCP, indicate the interaction between the two target molecules.

In these various embodiments, the RCPs may be detected by detecting their tag sequences (e.g. unique identifier sequences), or combinations thereof.

In an embodiment the relative levels of the first, second and third RCPs, and optional fourth RCP, respectively indicate the relative levels of the two target molecules and the proportion of each of the two target molecules interacting with each other.

In an embodiment the target molecules are proteins. In another embodiment, the first target molecule is a protein and the second target molecule is a modifying group on the protein (e.g. a post-translational modification (PTM).

In a second aspect, provided herein is a kit for detecting two target molecules in a sample, and detecting an interaction between the two target molecules, the kit comprising:
(i) a first probe or proximity probe pair for detection of a first target molecule, and a second probe or proximity probe pair for detection of a second target molecule, wherein said probes each comprise a binding domain capable of binding directly or indirectly to their target molecule and a nucleic acid domain, and wherein the first and second probe, or one of the probes of the first proximity probe pair and one of the probes of the second proximity probe pair, together form a third proximity probe pair for detection of the interaction between the first and second target molecules; and
(ii) first and second nucleic acid nucleic acid reagents, wherein the first reagent is capable of hybridising to the nucleic acid domain of the first probe or one of the probes of the first proximity probe pair and the second reagent is capable of hybridising to the nucleic acid domain of the second probe or one of the probes of the second proximity probe pair, optionally wherein the first and/or second reagent is further capable of hybridising to the nucleic acid domain of the other member of the third proximity probe pair; and
(iii) optionally a third nucleic acid reagent which is capable of hybridising to the nucleic acid domains of the third proximity probe pair.

In an embodiment, the nucleic acid reagents are substrate molecules capable of giving rise to distinguishable nucleic acid products.

### DETAILED DESCRIPTION

The present method provides a method for detecting two target molecules in a sample, and detecting an interaction between the two target molecules. As defined and described further below, more precisely the method detects proximity between two target molecules, and this proximity may indicate that the molecules are interacting. The method enables concurrent detection of both target molecules individually, and of their proximal location (e.g. complexes of the two target molecules). The method may thus be seen as a method of profiling an interaction.

For detection of at least the interaction between the two target molecules, the present method relies on the principle of ‘proximity probing’, wherein each target molecule is bound by a probe. Interaction of the two target molecules brings the probes into proximity, enabling an interaction between the two probes which causes a signal to be generated. Proximity probing may also be used to detect the individual target molecules.

Effectively, proximity probes or pairs of proximity probes, each specific for one of the two target molecules which may be interacting, are used in conjunction with additional ancillary reagents, which interact with the nucleic acid domains of the proximity probes to generate signals, which are indicative not only of the interaction, but also of the individual members of the interaction. By using additional reagents, the same, or common, proximity probes or members of a proximity pair may be used to generate different signals, for the two target molecules and their interactions. This allows the three separate determinations (i.e. three separate assay reactions, as outlined above) to be made in the context of the same assay, and furthermore allows these to be simultaneously revealed, in a manner which is readily accessible to multiplexing. In particular embodiments, the same probes which are used individually to detect the individual target molecules separately are also used together as a proximity probe pair to detect the target interaction. Thus, one particular probe may be used both to detect a target molecule individually, and also, in conjunction with another probe used to detect the other target molecule individually, to detect the target interaction. In other words, in such an embodiment one probe is used in the first and third assay reactions, and another probe is used in the second and third assay reactions. A proximity probe pair may thus be provided, the individual probe members of which are shared, or common, between the assay reactions used to detect a target molecule individually and the interaction.

Proximity assays may be performed in homogenously (i.e. in solution) or in a solid phase formats, and the present methods may be carried out in the context of any format of proximity assay which uses padlock probes, including both in-solution and solid phase formats, or mixed formats.

The two target molecules in the sample are different molecules (i.e. different species of molecule). That is to say the method is not suitable for detecting homomeric interactions. The target molecules may be any type of biomolecules, and the two target molecules may be different types of biomolecule. Thus, a target molecule may be for example a protein or a nucleic acid molecule, e.g. a DNA molecule or an RNA molecule.

In the context of a nucleic acid molecule, particularly a DNA molecule, the target molecule may be a particular nucleotide sequence within a much larger molecule, e.g. it may be a particular DNA sequence within a genome or chromosome. When one of the target molecules is a DNA molecule, it may be a natural or synthetic DNA molecule. A target DNA molecule (or target DNA sequence) may be coding or non-coding DNA, for example it may be genomic DNA, or may be derived from genomic DNA, e.g. it may be a copy or amplicon thereof, or it may be cDNA or an amplicon or copy thereof, etc.

When one of the target molecules is an RNA molecule, it may be an RNA molecule in a pool of RNA or other nucleic acid molecules for example genomic nucleic acids, whether human or from any other source, from a transcriptome, or any other nucleic acid (e.g. organelle nucleic acids, i.e. mitochondrial or plastid nucleic acids or viral nucleic acids), whether naturally occurring or synthetic. The target RNA molecule may thus be or may be derived from coding (i.e. pre-mRNA or mRNA) or non-coding RNA sequences (such as tRNA, rRNA, snoRNA, miRNA, siRNA, snRNA, exRNA, piRNA and long ncRNA). Alternatively, the target RNA molecule may be genomic RNA, e.g. ssRNA or dsRNA of a virus having RNA as its genetic material. Notable such viruses include Ebola, HIV, SARS, SARS-CoV2, influenza, hepatitis C, West Nile fever, polio and measles. Accordingly, a target RNA molecule may be positive sense RNA, negative sense RNA, or double-stranded RNA from a viral genome, or positive-sense RNA from a retroviral RNA genome. Where the target molecule is an RNA molecule, the method may comprise a preliminary step of generating a cDNA copy of the target RNA molecule.

Most commonly, the target molecules are both proteins. By ‘protein’ is here meant any amino acid-based biomolecule, and thus a target protein may be any polypeptide, oligopeptide or peptide comprising sufficient amino acids to be recognised by a probe used according to the invention. The protein may be a natural (wild type) protein from a human or other organism, or it may be a synthetic protein (e.g. a fusion protein), a protein fragment or a modified protein or mutant protein.

Thus, the method is most commonly for detecting the two proteins and the protein-protein interaction between them. In other embodiments however, the method may be used to detect interactions between a protein and another type of molecule, for example a nucleic acid, e.g. protein-DNA or protein-RNA interactions.

In another embodiment the two target molecules are a protein and a post-translational modification (PTM) of the protein. In this case the protein itself (modified and unmodified) is detected and the post-translationally modified version of the protein is separately detected, enabling quantification of the proportion of the protein which is post-translationally modified in the manner of interest. Any post-translational protein modification can be detected according to the method of the invention, e.g. alkylation (such as methylation), acetylation, glycosylation, phosphorylation, lipidation, ubiquitination etc.

Thus, more generally, the two target molecules may be a protein and a modifying group on the protein, or in other words, between a protein and a non-protein (non-amino acid) chemical group. Such a modifying group may be any group that may be recognised by a binding molecule (i.e. by a molecule used as, or to form, the binding domain of a proximity probe).

Analogously, the two target molecules may be a nuclei acid (e.g. RNA or DNA), or any biomolecule, and a modifying group on that nucleic acid or other biomolecule.

For example, a first target molecule may be a protein, DNA or RNA, and the second target molecule may be a lipid, carbohydrate, phosphate, alkyl, acetyl group etc, without limitation, which can be recognized by a binder.

The interaction between the two target molecules is an interaction in which the two target molecules come together. At its most general, this includes the proximal location of two molecules together. An interaction includes a binding, or physical connection between two molecules. Generally, the interaction is a direct interaction, whereby the two molecules bind to one another, though it may be an indirect interaction whereby the two target molecules are joined via a connecting molecule. In any event, in order for the interaction to be detected the two target molecules must be in sufficiently close proximity that the proximity probes bound to them are able to interact with each other. The interaction may be a covalent interaction, but is generally a non-covalent interaction.

Whilst conventionally an interaction denotes a physical interaction, directly or indirectly between two molecules, it will be understood that a proximity assay using proximity probes may simply detect the close proximity of two molecules, even if they are not in complex with one another or actually physically interacting. Thus, the term “interaction” as used herein includes a physical proximity between two target molecules, such that the two molecules when in proximity may be detected using a proximity probe pair. It is not required that there is binding, or a physical linkage or connection, between the two molecules. It suffices that the two target molecules are located in proximity to one another. Alternatively, in all instances where an “interaction” is referred to, this may be replaced by a reference to an interaction or proximity between the two target molecules. As indicated above, proximity in this context means that the two target molecules are sufficiently close to one another to enable them to be detected by a proximity assay. For example, this may in practice mean that they lie, or are located, within a distance of no more than 100, 90, 80 or more particularly 70 nm of each other, e.g. 10-80, 20-80, 20-70, 30-70, 40-70 nm of each other etc.

The sample on which the method of the invention is performed may be any sample which contains the target molecules. It may be a biological sample, e.g. a research sample or a clinical sample. The method of the invention may thus be used as a research tool or a diagnostic tool. The sample may be any type of biological sample, e.g. a cell or tissue sample, a fluid sample, a cell lysate, etc. The sample may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue green algae, fungi, bacteria, protozoa etc., or a virus. The cells may be for example human cells, avian cells, reptile cells etc., without limitation.

Representative samples thus include whole blood and blood-derived products such as plasma, serum and buffy coat, blood cells, urine, faeces, cerebrospinal fluid or any other body fluids (e.g. respiratory secretions, saliva, milk, etc.), tissues, biopsies, cell cultures, cell suspensions, conditioned media or other samples of cell culture constituents, etc. The method finds particular utility in histology and analysis of tissue and cell samples.

The sample may be freshly prepared, or may be pre-treated in any convenient or desired way to prepare for use in the method, for example by cell lysis or purification, isolation of the nucleic acid, etc. The sample may thus be processed as necessary, e.g. cells or tissue may, as required depending on the target molecules and other relevant factors, be fixed or permeabilised. Accordingly, fresh, frozen or fixed cells or tissues may be used, e.g. FFPE tissue (Formalin Fixed Paraffin Embedded). The present method is particularly advantageous as it is able to detect target molecules, and an interaction between them, at very low concentration.

In one embodiment, the sample is contacted with a first proximity probe for detection of the first target molecule and a second proximity probe for detection of the second target molecule. In another embodiment the sample is contacted with a first proximity probe pair for detection of the first target molecule and a second proximity probe pair for detection of the second target molecule. A single proximity probe for detection of the first target molecule may be used in combination with a proximity probe pair for detection of the second target molecule, but generally both target molecules will be contacted with either a single proximity probe or a proximity probe pair.

The proximity probes each comprise a binding domain and a nucleic acid domain. The binding domain is capable of binding directly or indirectly to the target molecule. In one embodiment the binding domain of each proximity probe specifically binds the target molecule it is designed to detect. In other words, it is a primary binding partner for the target molecule. In another embodiment it binds indirectly, via an intermediary binding partner, which is itself bound to the target molecule, as illustrated in FIG. 6 for example. In this case, the binding domain is a secondary binding partner for the target molecule, capable of binding specifically to a primary binding partner, itself bound to the target molecule.

The nature of a binding domain is dependent on the type of target molecule it is designed to detect. The binding domain may be any affinity binding partner for the target molecule, that is any entity capable of binding specifically to the target molecule. When the target molecule is a protein, the binding domain is generally an antibody or antigen-binding fragment or derivative thereof which is specific for the protein of interest. Examples of suitable antibody fragments and derivatives include Fab, Fab′, F(ab′)₂ and scFv molecules.

A Fab fragment consists of the antigen-binding domain of an antibody. An individual antibody may be seen to contain two Fab fragments, each consisting of a light chain and its conjoined N-terminal section of the heavy chain. Thus, a Fab fragment contains an entire light chain and the VHand CH1 domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules. ScFv molecules are synthetic constructs produced by fusing together the variable domains of the light and heavy chains of an antibody. Typically, this fusion is achieved recombinantly, by engineering the antibody gene to produce a fusion protein which comprises both the heavy and light chain variable domains.

Whilst binding domains based on the antigen-binding site of an antibody are typically used in proximity probes, the use of other binding domains is not precluded, including for example those based on receptor-ligand or other binding pairs, or aptamers etc.

As is well known in the art, when a pair of proximity probes is used to detect a single protein, the two binding domains bind at different sites, for example the two antibodies in the pair (or antibody derivatives) bind the protein at different epitopes.

When the target molecule is a nucleic acid molecule, the binding domain is generally also a nucleic acid molecule. In this instance the binding domain nucleic acid molecule is at least partially single-stranded, and the single-stranded region of the binding domain is, or comprises a region which is, complementary to the target molecule, such that it specifically hybridises to it. When the binding domain is a nucleic acid molecule it may be any nucleic acid, including a DNA or an RNA molecule, or any derivative thereof, but typically DNA.

The nucleic acid domain of a proximity probe may similarly be any nucleic acid molecule, but typically it will be a DNA domain. The nucleic acid domain is at least partially single-stranded. That is, the nucleic acid domain is either a single-strand, or comprises at least one single-stranded region. The single-stranded regions comprise the binding or hybridisation sites for nucleic acid reagents etc, or, in other words, comprise regions of complementarity to another nucleic acid molecule with which they are designed to hybridise, or to bind to. When the binding domain of the probe is an antibody or other non-nucleic acid molecule, the binding domain is conjugated to the nucleic acid domain. When the binding domain is a nucleic acid, the probe may be a single nucleic acid comprising the binding domain at one end and the nucleic acid domain at the other, optionally joined by a linker nucleic acid sequence. Alternatively, the binding domain nucleic acid and the nucleic acid domain may both be conjugated to a core group which is not a nucleic acid molecule.

The nucleic acid domains of each proximity probe are or comprise unique nucleotide sequences. Typically, each nucleic acid domain comprises at least one unique nucleotide sequence, or one nucleic acid sequence by means of which it may be detected or distinguished. By ‘unique’ is meant that none of the other proximity probes used in the method carries the same nucleotide sequence in its nucleic acid domain. In the alternative, the nucleic acid domain may comprise a tag sequence, by which it may be distinguished from the nucleic acid domain of another proximity probe. The tag sequence may be used to detect the nucleic acid domain of the proximity probe, and hence the target molecule to which it has bound. Furthermore, a tag sequence may provide a binding site for a nucleic acid reagent which is used in the assay reactions.

The nucleic acid(s) in the proximity probes may be arranged in any orientation. For instance, when the binding domain is conjugated to a single-stranded nucleic acid, the binding domain may be conjugated to either the 3′ or 5′ end of the nucleic acid. Similarly, in other probe designs the nucleic acid domains may be orientated in either direction, as convenient.

The first and second proximity probe, or where proximity probe pairs are used, one of the first proximity probe pair and one of the second proximity probe pair, form an additional proximity probe pair. If a single proximity probe is used to detect one of the target molecules and a pair of proximity probes is used to detect the other target molecule, the additional proximity probe pair is formed by the single proximity probe and one of the pair of proximity probes. In the case that a single proximity probe is used to detect each of the individual target molecules, this additional probe pair is the only probe pair used in the method. In the case that a single proximity probe is used to detect one target molecule and a proximity probe pair is used to detect the other target molecule, the additional probe pair is the second probe pair used in the method. In the case that a first and second proximity probe pair are used to detect the first and second target molecules, the additional probe pair is the third probe pair used in the method.

Nonetheless, for simplicity, the probe pair formed between probes which recognise the first and second target molecules is referred to herein as the third probe pair, in respect of all reaction set ups including those embodiments in which single proximity probes are used to detect the first and second target molecules individually. The single proximity probes in the discussion above are, in effect, single probes, since they are not used as proximity probes when used singly.

At its most general, the method encompasses performing first and second assay reactions to detect the first and second target molecules by detecting the nucleic acid domains of the probes by any known or convenient means. The third assay reaction detects the interaction between the two target molecules. In the third assay reaction a nucleic acid product is generated from the interaction of the nucleic acid domains of the third proximity probe pair, and is detected to detect the interaction. This nucleic acid product is conveniently referred to as the third nucleic acid product (since it arises from the third proximity probe pair). The first and second assay reactions may also, in some embodiments, result in the generation of first and second nucleic acid products respectively.

Since, the first and second assay reactions may include the simple detection of the nucleic acid domain of a probe, this may be performed using any convenient or desired method for detecting a nucleic acid. In this regard, as noted above, the first and second probes which are used for the detection of the first and second target molecules comprise a unique sequence or a tag sequence, which may be detected in order to detect the domain. Thus, a detection probe which binds specifically to the unique/tag sequence may be used, and a wide variety of such probes for detecting specific nucleic acid sequences are known in the art. These range from simple hybridisation probes, which may be directly labelled with a detectable label, or molecular beacons or such like. Alternatively, the unique or tag sequence of a probe can be detected by various sequencing methods.

More complex labelling systems based on hybridisation probes are also known and widely used in the art, for example “sandwich-type” systems where secondary or further detection probes are used, which hybridise to multiple binding sites on a primary detection probe, in order to amplify the number of labelling sites etc. Further, nucleic acid detection assays are known, which involve nucleic acid reactions such as extension, ligation, or cleavage to generate nucleic acid products which are generated. Such products may be generated from the nucleic acid domains themselves, for example, they may be cleaved to generate a product which is detected or ligated (to one another or to a further nucleic acid) or extended by a polymerase catalysed extension reaction, or they may serve to generate a product from another nucleic acid molecule with which the nucleic acid domain(s) interact(s), for example a primer which hybridises to the nucleic acid domain, which may be extended by a polymerase-catalysed reaction, or one or more oligonucleotides which are ligated using the nucleic acid domain as a ligation template. Further, an oligonucleotide may hybridise to the nucleic acid domain to generate a cleavage site, e.g. for a restriction enzyme etc.

As noted above, proximity assays are well known and used in the art to detect interactions between molecules, and individual target molecules; in the latter case, recognition of an individual target molecule relies on dual recognition of the target by each of the two members of a proximity probe pair. The nucleic acid domains of a pair of proximity probes may interact with each directly (e.g. they may hybridise to each other directly, and may for example be extended using the other domain as the extension template) or they may be ligated together when both hybridise to a common ligation template). They may also interact indirectly, for example when both interact with one or more other common nucleic acid molecules, for example both nucleic acid domains may template the ligation of added oligonucleotides, such as for example a padlock probe (see FIGS. 5, 6, 7 and 8 for example), or one nucleic acid domain may carry a padlock probe which targets the other nucleic acid domain (which acts as the ligation template), such that a proximity event is required in order to detect the target molecule or interaction by that padlock probe (see for example padlock C in FIG. 1, and FIG. 2, 3, 4 or 13).

Thus, generally speaking, a detection reaction to detect a nucleic acid domain of a probe, or to detect the interaction between the nucleic acid domains of proximity probes, involves the use of another nucleic acid molecule (e.g. an oligonucleotide) which interacts with, or hybridises to the nucleic acid domain(s). Thus, the detection reaction, or more generally the first, second, and third assay reactions, may involve the use of a nucleic acid reagent which hybridises to the nucleic acid domain(s) of the probes.

As noted above, the nucleic acid reagent may be a simple detection probe, the hybridisation of which to the nucleic acid domain of the first or second probe may be detected in order to detect the first or second target molecule individually. However, more complex nucleic acid-based signal generation systems are known in the art, to increase the signal, and aid detection, which may increase the sensitivity of the method. In such systems further nucleic acid reagents (e.g. oligonucleotides) are used to build up, or generate, a nucleic acid product that can be detected. Any such DNA-based signal amplification system can be used. Such methods include, as well as methods based on amplification or polymerase chain extension reactions and/or ligation reactions, methods in which hybridisation of oligonucleotides is used to build up a large nucleic acid product. These include hybridisation chain reaction (HCR), where short single-stranded oligonucleotides (HCR monomers) are hybridised together to build up a long, nicked doubled-stranded nucleic acid chain, and also branched DNA amplification reactions, where a sequence of intermediate hybridisation probes is used, which hybridise to each other to provide multiple hybridisation sites for further probes, such that a branched nucleic acid structure is built up, ultimately providing multiple binding sites for a multiplicity of labelled detection probes.

Thus, a template, or scaffold molecule may be provided which hybridizes to its target nucleic acid sequence in the nucleic acid domain of a probe (e.g. to a tag or unique sequence in the domain), and which comprises multiple binding sites for further hybridization probes. These may comprise detection sequences to which a labelled detection probe may bind, or they may comprise binding sites for further hybridization probes etc. Thus, a “layered” or “branched” structure may be made up, composed of multiple hybridization probes. The hybridization probes, or a subset thereof (e.g. the last or final hybridization probes added to the structure), may comprise a detection sequence. The nucleic acid product may in such an embodiment be referred to as a hybridization assembly. This is exemplified by the RNAscope™ technology, as described in WO2011/094669 for example. Whilst RNAscope™ was developed for in situ hybridisation for detection of RNA, it exemplifies the principle of using sandwich-type, or intermediate, hybridisation probes each providing multiple binding sites for labelled detection probes, to generate a detectable nucleic acid product comprising multiple labels.

Such amplification methods which generate a nucleic acid product may be adapted for use in proximity-based assays reactions. For example, a proximity HCR method is described in WO2015/118029 which describes a system where the interaction between the nucleic acid domains of proximity probes opens a secondary structure in a nucleic acid domain to reveal an initiator for an HCR reaction. A separate external activator molecule may be used, which binds to a nucleic acid domain, to open up a secondary structure (e.g. hairpin) which in turn opens up the nucleic acid domain of the other proximity probe to reveal the HCR initiator. The HCR initiator then opens up the hairpin of the first HCR monomer, to initiate the HCR reaction between 2 sets of HCR monomers. The HCR monomers may be labelled, or may be provided with detection sites for the binding of labelled detection probes, to allow the HCR product to be detected.

Similarly, as is known in the context of branched DNA amplification technology, proximity binding can be used for the binding of the primary scaffold molecule on which the branched DNA structure is built. Thus, the nucleic acid domains of proximity probes may, when brought together in proximity by the binding of a proximity probe pair to the first and second target molecules, allow the binding of a scaffold oligonucleotide for a branched DNA amplification reaction.

Accordingly, it will be seen from the above that in an embodiment, the nucleic acid reagent may be a substrate from which, or using which, a nucleic acid product may be generated.

As indicated above a separate substrate may be used for each of the three assay reactions, but this is not necessary and one or both of the substrates for the first and second reactions may be used also in in the third assay reaction. It will be understood in this regard, that in such a method, the substrate will be used in a different way, such that a different nucleic acid product is generated.

In the method, the probes may be contacted with at least first and second nucleic acid substrate molecules. This contacting step may take place before, during or after the probes are applied to the sample, as necessary or preferred depending on the specific set-up used for the method. The first substrate molecule hybridises with the nucleic acid domain of the first proximity probe, or one of the probes of the first proximity probe pair, and the second substrate molecule hybridises with the nucleic acid domain of the second proximity probe, or one of the probes of the second proximity probe pair.

More particularly, the first nucleic acid substrate specifically hybridises to the nucleic acid domain of the first proximity probe, or one of the probes of the first proximity probe pair. As noted above, each nucleic acid domain comprises at least one sequence by means of which it may be distinguished, for example at least one unique nucleotide sequence or at least one tag sequence. The first nucleic acid substrate comprises one or more cognate sequences which is, or together are, complementary to one of the distinguishable or unique sequences in the nucleic acid domain of the first proximity probe or in one of the nucleic acid domains of the first proximity probe pair. Equivalently, one or more distinguishable or unique sequences in the second nucleic acid substrate are complementary to, and specifically hybridise to, one of the distinguishable or unique sequences in the nucleic acid domain of the second proximity probe or one of the nucleic acid domains of the second proximity probe pair. In other words, the substrate comprises a binding site which is complementary, and capable of hybridising to a cognate substrate-binding site in the nucleic acid domain.

In a particular embodiment the first and second nucleic acid substrates are padlock probes (i.e. first and second padlock probes, respectively). The operation of the method using padlock probes is described in more detail below. However, the skilled person will understand that padlock probes may be replaced by other types of substrate, such as a component of an HCR reaction or branched DNA amplification reaction as described above.

The choice of substrate, or detection modality, may depend on the nature of the target molecules, and the sample etc. For example, for in situ detection methods, where localised detection of the target molecules is desirable, the generation of nucleic acid products, such as RCPs for example, may be desirable. Thus, padlock probes and RCA-based detection are convenient in such a setting. However, other detection methods may be used, according to choice. Such methods may include, for example, PCR-based methods. For non-in situ detection, it may be convenient to detect the target molecules (or more particularly the nucleic acid domains of the probes, and/or the nucleic acid product(s)) by sequencing or PCR-based methods (e.g. qPCR). However, this nature of the detection, and hence of the nucleic acid products which are generated, or detection probes etc., which are used, is not limited. For example, nucleotide detection methods such as STORM (Rust et al., Nature Methods 2006, 3(10), 793-795) or DNA Paint (Schnitzbauer et al., 2017, Nature Protocols, 12, 1198-1228) may be used.

As noted above, a padlock probe may be defined as a circularisable probe. The use of padlock or circularisable probes is well known in the art, including in the context of RCA reactions. A circularisable probe comprises one or more linear oligonucleotides which may be ligated together to form a circle. Thus, the principles of padlock probing are well understood and the design and use of padlock probes is known and described in the art. A padlock probe is typically a linear circularisable oligonucleotide which hybridises to its target nucleic acid sequence or molecule in a manner which brings 5′ and 3′ ligatable ends of the probe into juxtaposition for ligation together, either directly, or indirectly in which case a gap is located between the ligatable ends of the probe when it is hybridised to its target sequence. By ligating the hybridised 5′ and 3′ ends of the probe, the probe is circularised. It is understood that for circularisation (ligation) to occur, the ligatable 5′end of the padlock probe has a free 5′ phosphate group. Where there is a gap, this is filled either by a gap oligonucleotide (which can be viewed as a part of the padlock probe), of by extension of the hybridized 3′ end of the padlock probe.

To allow the juxtaposition of the ends of the padlock probe for ligation, the padlock probe is designed to have the target-binding sites at its 5′ and 3′ ends. That is, the regions of complementarity which allow binding of the padlock probe to its target lie at the ends of the padlock probe. The region of the padlock probe which connects, or lies between, the target-complementary ends, and which is not capable of hybridizing to its target molecule, is commonly referred to as a backbone region.

To allow ligation, the 3′ and 5′ ends which are to be ligated (the “ligatable” 3′ and 5′ ends) are hybridised to a target sequence, which acts as the ligation template. Herein the target sequence for a padlock probe (i.e. a region in the target molecule complementary to a target-binding region of a padlock probe) is generally referred to as a padlock binding site. The term “hybridisation sequence” in relation to a padlock probe more generally includes a sequence which is capable of hybridizing to a padlock probe at any site in the padlock probe, and this includes to a complementary sequence in the backbone region, as well as to the target-binding regions of the padlock probe.

The binding of the padlock probe to its target sequence brings its ends into said juxtaposition. Where the complementary binding sites in the target molecule or sequence lie directly adjacent (or contiguous) to one another, the ends of the padlock probe will hybridise directly adjacent to each other (i.e. with no gap) and may be ligated to each other directly. Thus, in this case the ligatable ends of the probe are provided by the actual ends of the probe. In the case of a gap-fill padlock probe, the target binding regions at the ends of the padlock probe do not hybridise to adjacent binding sites, but rather to non-adjacent (non-contiguous) binding sites in the target molecule. In such an arrangement, the 5′ ligatable end of the probe is provided by the actual 5′ end of the probe. However, the ligatable 3′ end of the probe may be generated by extension of the hybridized 3′ end of the probe, using the target sequence as extension template to fill the gap between the hybridized ends of the probe. The extension reaction brings the extended 3′ end of the probe into juxtaposition for ligation. In this case, the ligatable 3′ end of the probe is thus the extended 3′ end of the probe. In another embodiment, juxtaposed ligatable 5′ and 3′ ends are provided by a gap oligonucleotide which hybridizes in between the two ends of the padlock probe and is ligated thereto to form a circle. In this case, there are two ligation junctions. This can be seen as a form of 2-part padlock probe. In still another configuration, a 2-part padlock probe may have 2 target sequences, each of which act as ligation templates for the probe, where the 5′ end of one part (a first circularisable oligonucleotide) hybridizes in juxtaposition for ligation to the 3′ end of the other part (a second circularisable oligonucleotide), and both parts are ligated together to form a circle (see FIGS. 1B, 1C, 5, 6, 7, and 8 for example).

The term “hybridisation” or “hybridises” as used herein generally refers to the formation of a duplex between nucleotide sequences which are sufficiently complementary to form duplexes via Watson-Crick base pairing, or any analogous base-pair interactions. Two nucleotide sequences are “complementary” to one another when those molecules share base pair organization homology. Hence, a region of complementarity in a molecule or probe or sequence refers to a portion of that molecule or probe or sequence that is capable of forming a duplex. Hybridisation does not require 100 % complementarity between the sequences, and hence regions of complementarity to one another do not require the sequences to be fully complementary, although this is not excluded. Thus, the regions of complementarity may contain one or more mismatches. Accordingly, “complementary”, as used herein, means “functionally complementary”, i.e. a level of complementarity sufficient to mediate a productive hybridisation, which encompasses degrees of complementarity less than 100 %. The degree of mismatch tolerated can be controlled by suitable adjustment of the hybridisation conditions. Those skilled in the art of nucleic acid technology can determine duplex stability empirically considering a number of variables including, for example, the length and base pair composition of the respective molecules or probe oligonucleotides, ionic strength, and incidence of mismatched base pairs, following the guidance provided by the art. Thus, the design of appropriate probes, and binding regions thereof, and the conditions under which they hybridise to their respective targets is well within the routine skill of the person skilled in the art.

A region of complementarity, such as for example to a target sequence in the binding region of a padlock probe, or between a detection sequence and a detection oligonucleotide, or a RCA primer to the circularised padlock probe etc., may be at least 6 nucleotides long, to ensure specificity of binding, or more particularly at least 7, 8, 9 or 10 nucleotides long. The upper limit of length of the region is not critical, but may for example be up to 50, 40, 35, 30, 25, 20 or 15 nucleotides. A complementary region may thus have a length in a range between any one of the lower length limits and upper length limits set out above. In the case of a padlock probe, the length of an individual target-binding region may be in the lower ranges, so that the total length of the two binding regions when hybridised to their target is within the upper ranges. For example, an individual target binding region may be 8-15, e.g. 10-12 nucleotides, so that the total hybridised length is 16-30 nucleotides long, e.g. 20-24. It may be desirable, within the constraints of conformation of the probes, and spacing of the domains, and desired or favoured hybridisations, to minimise the total length of a padlock probe to minimise the size of the circle which is subjected to RCA, and hence to minimise the lengths of the complementary regions where possible.

In the embodiment in which the first and second nucleic acid substrates are padlock probes, the nucleic acid domain of the first proximity probe, or one of the probes of the first proximity probe pair (the “first nucleic acid domain”), comprises a padlock binding site complementary to the target binding sequences at the 5′ and 3′ ends of the first padlock probe. Equivalently, the nucleic acid domain of the second proximity probe, or one of the probes of the second proximity probe pair (the “second nucleic acid domain”), comprises a padlock binding site complementary to the target binding sequences at the 5′ and 3′ ends of the second padlock probe. The term “padlock binding site” as used herein refers simply to a sequence in a nucleic acid to which a padlock probe hybridises in the method. In particular, the term refers to a sequence to which a padlock probe constituting the first or second nucleic acid substrate molecule binds.

Once the probes have been contacted with the first and second nucleic acid substrate molecules and the sample (in whichever order those steps are performed) two assay reactions are performed to detect the first and second target molecules. The first target molecule is detected by generating a first nucleic acid product from the first nucleic acid substrate, and the second target molecule is detected by generating a second nucleic acid product from the second nucleic acid substrate. The first and second nucleic acid products are detected (as further discussed below), thus detecting the first and second target molecules. The first and second assays, to detect the first and second target molecules, are generally performed concurrently, but depending on the specific set up of the method used may be performed sequentially.

When the first and second nucleic acid substrates are padlock probes, the assays comprise circularisation and, generally speaking but not necessarily, amplification of the padlock probes. As set out above, when the padlock probe hybridises to the padlock binding site in its target nucleic acid domain, its target-binding regions (i.e. its 3′ and 5′ ends which hybridise to the nucleic acid domain) may bind to the padlock binding site directly adjacent to one another or with a gap in between, which is filled in before ligation to circularise.

As mentioned above, other types of nucleic acid substrate (other than padlock probes) may be used if desired, in which case the first and second assay reactions selected are appropriate for the types of nucleic acid substrate used.

A third assay reaction is also performed to detect the interaction between the first and second target molecules using the third proximity probe pair. When the first and second target molecules are interacting the members of the third proximity probe pair directly or indirectly interact. The third assay reaction generates a third nucleic acid product indicative of the interaction between the probes of the third proximity pair. Detection of the third nucleic acid product corresponds to detection of the interaction of the probes of the third proximity pair, and thus detection of the interaction between the first and second target molecules.

As mentioned, the interaction between the members of the third proximity probe pair may be direct. That is to say, the members of the third proximity probe pair may directly hybridise to each other. In this embodiment, the interaction of the probes of the third proximity probe pair forms the basis for a proximity extension assay (PEA). PEA methods are well known in the art and described in e.g. WO 03/044231 and WO 2004/094456. In this case the ends of the reporter nucleic acids of the third proximity probe pair (i.e. the ends of the reporter nucleic acids distal to the binding domains) are complementary to allow them to hybridise to one another.

Generally, however, the probes of the third proximity probe pair interact indirectly, i.e. via one or more intermediate nucleic acid molecules or oligonucleotides which hybridise to both probes. In particular, the third proximity probe pair may be contacted with a third nucleic acid substrate molecule which hybridises to both probes in the pair. A third nucleic acid product is then generated from the third nucleic acid substrate molecule. In a particular embodiment the third nucleic acid substrate molecule is a padlock probe.

As noted above, the padlock probes used herein may be provided in one or two parts. That is to say, generally, a padlock probe is provided in a single part, i.e. as a single linear nucleic acid molecule with target-binding regions at both ends, such that the two ends of the probe bind the target sequence adjacent to one another enabling the probe's circularisation. The first and second nucleic acid substrate molecules are conveniently in many cases one-part padlock probes, but as will be clear from the more detailed description below, it is not precluded that they may be 2-part probes with a gap oligonucleotide configuration (see FIG. 3B for example). However, the padlock probes used in certain embodiments, particularly as the third nucleic acid substrate molecule, may be provided in two parts in other configurations. A padlock probe may be provided in two parts when it is to bind two separate target nucleic acid molecules, e.g. two separate nucleic acid domains (see FIGS. 1B and 1C for example). As noted above, in one such embodiment, it is provided as two linear nucleic acid molecules (circularisable oligonucleotides) each of which bind both target nucleic acid molecules (i.e. both parts of the padlock probe have one end which binds one target nucleic acid molecule and one which binds the other). The two parts of the padlock probe bind the two target nucleic acid molecules such that on both target molecules the ends of the respective padlock probe parts are adjacent to one another, so that the two parts together have a circular conformation, and can be ligated to form a circular nucleic acid (either directly or indirectly, as described above).

The third nucleic acid substrate can thus be provided as a padlock probe in two parts. Alternatively, the third nucleic acid substrate can be provided in the form of a traditional one-part padlock probe. However, in this instance the probe must be provided pre-hybridised to one of the probes in the third probe pair, with its ends free to hybridise to the other probe of the pair to thus join the probes of the third probe pair in an indirect interaction. Thus, in such an embodiment, the third padlock probe comprises an anchor sequence in the backbone region between the target-binding ends of the padlock. The anchor sequence is a binding site for a complementary binding site in the nucleic acid domain of a proximity probe in the third proximity probe pair (e.g. the first proximity probe may comprise a hybridisation sequence in its nucleic acid domain which is capable of hybridising to the anchor sequence). A hybridisation sequence may thus be provided in the nucleic acid domain of a proximity probe which is capable of hybridising to an anchor sequence (or more generally a backbone sequence) in a padlock probe. It will thus be understood that the anchor sequence is simply a sequence which allows the third padlock probe to be hybridised to the nucleic acid domain of a proximity probe in a manner which leaves its ends free for target binding. It may alternatively be referred to as a capture sequence. The third padlock probe may be pre-hybridised to one of the proximity probes of the third proximity probe pair (e.g. first proximity probe) prior to performing the method (e.g. it may be supplied this way) or it may be pre-hybridised in use, e.g. by contacting the third padlock probe with the proximity probe prior to contact with the sample.

In another embodiment, the third nucleic acid substrate molecule is the first or second substrate molecule, that is to say a single substrate molecule acts to detect the first or second target molecule alone and the interaction between the target molecules. Such an arrangement may be achieved in a variety of ways.

For example, a padlock probe may be provided pre-hybridised to the first proximity probe, in complex with a blocking oligonucleotide which hybridises to the padlock probe's target binding regions. The padlock probe's target binding regions hybridise to the nucleic acid domain of the second proximity probe, with higher affinity than they hybridise to the blocking oligonucleotide. Thus, when the two target molecules interact the nucleic acid domain of the second proximity probe outcompetes and displaces the blocking oligonucleotide, and hybridises to the padlock probe. The padlock probe hybridises to one or both of the blocking oligonucleotide and the second nucleic acid domain with a gap in between its 3′ and 5′ ends. Thus, a gap sequence exists in the blocking oligonucleotide and/or second nucleic acid domain, which lies between the binding sites/hybridisation sequences (i.e. the complementary binding sites on the blocking oligonucleotide or second nucleic acid domain) which hybridise to the target binding ends of the padlock probe. Such a gap sequence provides a means by which the resulting circularisation product of the padlock probe may be distinguished. In other words, the gap sequence may provide or comprise an identifier sequence. For example, it may provide or comprise (or consist of) a barcode sequence or tag sequence. This gap may be filled by a gap oligonucleotide which is complementary to the gap sequence or by gap-filling extension of the hybridised 3′ end, as described above. Upon circularisation of the padlock probe the relevant gap sequence is incorporated, enabling distinction between padlock probe bound to the blocking oligonucleotide (corresponding to non-interacting target molecule) and padlock probe bound to the second nucleic acid domain (corresponding to interacting target molecule). This arrangement of the method is shown in FIG. 2.

More generally blocking oligonucleotides may be used in other formats and configurations of the method to control hybridisation and ligation of padlock probes. This may enable better control of unwanted non-specific background reactions. Thus, the target binding ends of a padlock probe used in any of the embodiments of the method described herein may be protected by a blocking oligonucleotide, which prevents the padlock probe from hybridising to its intended target until the blocking oligonucleotide is removed. Thus, the padlock probe may be provided, or used, in the form of a complex with a blocking oligonucleotide. As an example of such a format, the configuration presented in FIG. 1A or 3A could be modified to provide each of padlocks A, B and C with a blocking oligonucleotide hybridised to the target-binding ends of the padlocks. Similarly, the three padlocks depicted in FIG. 13 could be provided with blocking oligonucleotides. Removal may be displacement of the blocking oligonucleotide by the intended target, or by a separately added key oligonucleotide which is able to invade and displace the blocking oligonucleotide.

In another embodiment both target molecules are detected with a pair of proximity probes. In each probe pair, one probe has a nucleic acid domain with a pre-hybridised padlock probe and the other has a nucleic acid domain which comprises a padlock binding site with a gap sequence (which gap sequence may comprise a barcode/tag sequence). The padlock probe of each pair is able to bind to the nucleic acid domains of both its paired proximity probe and the proximity probe of the other pair which has the gap sequence. When the target molecules are not interacting, the padlock probe of each pair hybridises to the nucleic acid domain of the paired proximity probe; when the target molecules are interacting, the padlock probes interact with the free (i.e. non-padlock carrying) nucleic acid domain of the proximity probe of the other probe pair. As above, upon circularisation of the padlock probe the relevant gap sequence is incorporated, enabling distinction between padlock probe bound to its paired proximity probe (corresponding to non-interacting target molecule) and padlock probe bound to the proximity probe of the other pair (corresponding to interacting target molecule). This arrangement of the method of the invention is shown in FIG. 3B, which illustrates a gap oligonucleotide hybridised to the gap sequences.

Other configurations which use pairs of proximity probes to detect the individual target molecules are also possible. For example, FIG. 3A shows a variant of FIG. 1A, in which a first and second pair of proximity probes is used to detect both the first and second target molecules respectively in the first and second assay reactions. In this case, one of the proximity probes of the pair carries a pre-hybridised padlock probe which has target-binding sites (ends) specific for the nucleic acid domain of the other member of the proximity probe pair. When both probes of the first or second pair have bound to their target molecule, the padlock is able to hybridise to its target binding site in its paired proximity probe, and the circularised padlocks are detected in the first and second assay reactions. To detect the interaction between the two molecules, a third padlock probe is used in the third assay reaction. This may also be pre-hybridised to the nucleic acid domain of the proximity probe of one of the first and second pairs which carries the first or second padlock. The third padlock probe has target binding sites (ends) which are capable of hybridising to a separate binding site (hybridisation sequence) in the nucleic acid domain of a proximity probe of the other pair. When the two target molecules are in proximity (by virtue of being in an interaction with one another) and the first and second proximity probes pairs have both bound to their respective target molecule, the third padlock probe in one of the first or second pairs is able to hybridise to its target sequence in the other pair and the circularised padlock is detected in the third assay reaction. This is described in more detail below.

A configuration which functions similarly to that of FIG. 3A is shown in FIG. 13. This is based on the same concept of first, second and third proximity probe pairs being used in first, second and third assay reactions respectively, to detect first and second target molecules, and the interaction between them. However, in this case, the nucleic acid domains are differently designed; each are single stranded and in the proximity probes of the first and second pairs which are used to constitute the third pair, the nucleic acid domain is conjugated to the binding domain at an internal site (rather than at an end) such that it is able to provide two separate binding sites, one for binding the first or second padlock and the other for binding the third padlock. The padlocks may be provided prehybridised to the nucleic acid domains at their backbone/anchor sequences, leaving their target binding ends free to hybridise to the nucleic acid domain of the other member of the proximity probe pair. Further, the three padlocks may each be provided in complex with a blocking oligonucleotide hybridised to their target-binding free ends, as discussed above. The use of the blocking oligonucleotide may improve the performance of such a configuration, by allowing control of the hybridisation of the padlocks to their respective binding sites on the nucleic acid domains on the proximity probes. As indicated above, the blocking oligonucleotide may be removed by displacement by the target sequence of the padlock probe, or by a separately added key oligonucleotide which is able to invade and displace the blocking oligonucleotide.

A still further configuration in which pairs of proximity probes are used to detect individual target molecules is shown in FIG. 6. Here, the first and second proximity probe pairs are secondary reagents for detecting an individual target molecule, which bind to a primary binding partner itself bound specifically and directly to a target molecule. One of the proximity probes of a pair is used to detect and report on the molecule; this proximity probe has a nucleic acid domain which is detected by a padlock probe specific for that domain (namely the first and second padlock probes each specific for the probe from the first and second proximity probe pairs respectively). Thus, each of these proximity probes is used as an individual probe to detect the first and second target molecules in the first and second assay reactions respectively using first and second padlock probes. The third assay reaction uses the other member of the first and second proximity pairs, which together constitute the third proximity probe pair for the third assay reaction. In the configuration shown here, the third padlock probe is provided as a two-part probe, one end of each part of which hybridises to one of the nucleic acid domains of the third proximity probe pair, to bring the respective ends of the two parts into juxtaposition for ligation, using the nucleic acid domains of the third proximity probe pair as two separate ligation templates. In this way, three separate products are created, circularised padlock probes, from each of the first, second and third padlock probes. The circularised padlocks may be amplified by RCA, and as shown in FIG. 6, the RCA reactions may conveniently be primed by a nucleic acid domain of the proximity probes.

A modified version of the assay format of FIG. 6 is shown in FIG. 7. Here, the pair of proximity probes used to detect each of the two target molecules individually is replaced by a single probe, but in this case the probe carries two separate nucleic acid domains (i.e. wherein each separate nucleic acid domain is separately conjugated to the binding domain of the probe (the binding domain is depicted as an antibody in FIG. 7, but may be any binding domain for the analyte, or for a primary binding partner therefor). As shown in Figure each of the two separate nucleic acid domains is single-stranded, but all that is required is that the nucleic acid domain is at least partially single-stranded in the region that comprise binding sites for the padlock probe. Thus, in this version, there are two probes, each carrying two separate nucleic acid domains (4 oligonucleotides in total, corresponding to the 4 oligonucleotides constituting the nucleic acid domains of the two proximity probe pairs shown in FIG. 6). The first nucleic acid domain of the first probe is detected by a first padlock probe to detect the first target molecule (depicted as A). The first nucleic acid domain of the second probe is detected by a second padlock probe to detect the second target molecule (depicted as B). The probes (first and second probes) are also (and separately) used together as a proximity probe pair (the “third” proximity probe pair according to the terminology herein), and in this case the proximity interaction between the second nucleic acid domains of the first and second probes is detected using a third padlock probe to detect the interaction between the first and second target molecules (depicted as AB). The third padlock probe is provided as a two-part probe, one end of each part of which hybridises to one of the second nucleic acid domains of the third proximity probe pair, and the other end of each part of which hybridises to the other of the second domains, to bring the respective ends of the two parts of the padlock probe into juxtaposition for ligation, using the second nucleic acid domains of the third proximity probe pair as two separate ligation templates. In this way, three separate products are created, circularised padlock probes, from each of the first, second and third padlock probes. The circularised padlocks may be amplified by RCA, and as shown in FIG. 7, the RCA reactions may conveniently be primed by a nucleic acid domain of the proximity probes. The use of single probes with two different nucleic acid domains allows the probes to be used as primary binding reagents for the target molecules (rather than as secondary reagents as depicted in FIG. 6).

In particular embodiments the padlock probes used herein do not have secondary structure, and more particularly do not comprise intramolecular double-stranded regions or stem-loop structures. However, dumbbell probes, which do have secondary structure, are a particular sub-type of padlock probe which may be used. The dumbbell probe comprises two stem-loop structures, joined stem to stem, wherein one of the “loops” is not closed, but is open with free 5′ and 3′ ends available for ligation to each other. This “open loop” functions as the target-binding domain of the probe. The closed loop functions simply as a spacer to join the end of the duplex (stem). In other words, it can be seen as a padlock probe with a region of duplex formed between complementary sequences (regions) of the padlock. The region of duplex functions as a signalling domain to which an intercalating agent can bind. Thus the “open loop” of a dumbbell probe may comprise the target-binding regions of complementarity.

To perform the padlock probe-binding step the padlock probe is typically incubated with the proximity probes. As stated above, depending on the specific set-up of the method, this may be performed before the proximity probes are applied to (i.e. contacted with) the sample, in order to pre-hybridise the padlock probes to the proximity probes, or after the proximity probes have been applied to the sample, in which case the padlock probes are also applied to the sample. For a gap-fill padlock probe, dNTPs and polymerase may also be included. Conveniently a ligase may also be included during the padlock probe binding steps. The reagents may be added in a single reaction mix, or separately before or during the probe-binding step. To allow for probe-binding there may be an initial heating step, for example to denature a double-stranded nucleic acid molecule. The reagents are typically provided in a buffer, according to principles and procedures known in the art. For example, a buffer appropriate for the selected ligase enzyme may be selected.

The reaction mixture may be incubated in conditions appropriate to facilitate or enable padlock probe binding (the so-called “annealing” step). If there has been a preceding denaturation step this may involve a reduction in temperature. Conditions for these steps are known in the art, and are within the routine skill of the skilled practitioner in the art to select or design. For example, an annealing temperature of room temperature, or in the range of 20-40° C. may be used, e.g. 25-40° C., or 25-37° C. In one embodiment a higher temperature, e.g. 50-65° C. may be used, e.g. 53-60° C., or 55-60° C. If an elevated annealing temperature is selected, the annealing temperature may be reduced for the extension step if a gap-fill padlock is used. Again, the appropriate conditions can be selected according to what is known in the art, and the particular reagents, e.g. enzymes used. For example, after the initial annealing step, the temperature may be reduced to 28-40° C., e.g. 28-35, 30-35, 28-33, 30-33 or 30-32° C., etc.

For ligation of the padlock probes any convenient ligase may be employed, and representative ligases of interest include, but are not limited to, temperature sensitive ligases such as SplintR ligase (also known as PBCV-1 DNA ligase or Chlorella virus DNA ligase) bacteriophage T4 DNA ligase, bacteriophage T7 ligase, and E. coli ligase, and thermostable ligases such as Taq ligase, Tth ligase, Ampligase®, Pfu ligase and 9°N™ DNA Ligase.

Suitable conditions for ligation are known in the art, and any reagents that are necessary and/or desirable may be combined with the reaction mixture and maintained under conditions sufficient for ligation. It will be evident that the ligation conditions may depend on the ligase enzyme used in the methods of the invention. Thus, for example, Ampligase may be used, and the temperature may be increased for the ligation step. Alternatively, SplintR ligase may be used at room temperature.

Where temperature change or temperature control steps are required, the method may be performed in a thermal cycling instrument. This permits a ready control of the temperature changes. However, an advantage of the method is that extreme temperature changes are not necessary, and the method may for example be performed at room temperature, or at 20-37° C. for example. Probe binding and ligation steps may for example be performed at room temperature.

The conditions for the probe binding and ligation reactions may be optimised by routine experimentation according to principles known in the art. Thus, temperature, buffers, time of incubation, ramping speed etc. may be adjusted to find the optimal conditions.

Thus, first, second and third nucleic acid products may be generated, corresponding to the first target molecule alone, the second target molecule alone and the interaction between the two target molecules, respectively. The three nucleic acid products are distinguishable from each other by sequence. That is to say, each of the three products has or comprises a different sequence, i.e. a detection sequence, enabling distinction of the three nucleic acid products based on their sequences. The term “detection sequence” as used herein includes both the detection sequence as it occurs in the product (e.g. in a circularised padlock probe), and the complementary copy as it appears in the amplification product thereof. The three nucleic acid products may each have completely different sequences from which a unique detection sequence can be selected. Alternatively, each may have a shared backbone, differing only by an identifying detection sequence used for detection of each product. Accordingly, it will be understood from this that a detection sequence in the product corresponds to an identifier sequence as referred to above.

Accordingly, a padlock probe may carry an identifier sequence by which it may be detected and identified. That is a sequence which distinguishes it from other padlock probes, i.e. is a different sequence in different padlock probes. As noted above, this may be referred to as a unique identifier sequence. This may be e.g. a tag or barcode sequence. A tag sequence is essentially an identificatory sequence and hence may be regarded as equivalent to or synonymous with an identifier sequence. A barcode sequence is also an identificatory sequence, which conveniently may be detected by decoding the sequence, for example by sequencing, which may include sequencing by hybridisation and sequencing by ligation reactions, as well as conventional sequencing reactions e.g. sequencing by synthesis. Thus, more broadly a barcode sequence may simply be regarded as a sequence which identifies a nucleic acid molecule (e.g. a padlock probe) and which can be detected by detecting and identifying its sequence. The padlock probe may carry such an identifier sequence in its backbone region, or the identifier sequence may lie in the target binding region(s) of the padlock probe. In the latter case, then the identifier sequence will reflect (e.g. be the complement of) a sequence in the target of the padlock probe, i.e. an identifier (or tag) sequence in the nucleic acid domain of a proximity probe. Further, an identifier sequence in the backbone of the probe may lie in a region/sequence which is designed to hybridise to a nucleic acid domain, e.g. to a complementary padlock hybridisation sequence in a nucleic acid domain, for example an anchor sequence of a padlock probe. In this manner also, an identifier sequence may reflect an identifier sequence present in the nucleic acid domain of a proximity probe. Alternatively, or additionally, an identifier sequence may be incorporated into a circularised padlock probe by a gap-filling reaction, as described above. The gap sequence in a ligation template to which a padlock probe has hybridised (e.g. a nucleic acid domain or blocking oligonucleotide), between the hybridised ends of the padlock, may comprise or constitute an identifier sequence, the complement of which is incorporated into the circularised padlock probe. Thus, generally speaking an identifier sequence includes the complementary sequence. In certain embodiments, a padlock probe may contain more than one identifier sequence.

Analogously, identifier sequences may be incorporated into, or provided, in other nucleic acid substrates used to generate nucleic acid products, for example in primers, or HCR monomers, or oligonucleotides used in hybridisation-based branched DNA methods as described above.

Further, in the context of simple detection assays which use detection probes directly to detect the nucleic acid domains of first and second probes used for detection of the first and second target molecules individually, an identifier sequence may be present in the nucleic acid domain of the probe, and may be targeted by the detection probe.

The first, second and third assays are generally performed together, concurrently, though sequential performance may be possible in some embodiments.

Where the assays have been performed to generate nucleic acid products an amplification reaction may be performed to generate amplification products of or from each of the nucleic acid products. The amplification reaction can be performed in any suitable manner, and this may depend on the nature of the nucleic acid product. In some cases, the nucleic acid product is itself the result of a signal amplification reaction, e.g. HCR or branched chain amplification, and so may not need, or may not be suitable for, further amplification. However, further amplification is not necessarily precluded. For example, the nicks in an HCR product may be ligated and the ligated HCR product may be subject to further amplification, e.g. by a PCR reaction. Where the nucleic acid products are circular, they are advantageously amplified by rolling circle amplification (RCA). Any suitable amplification reaction known in the art may be used.

RCA utilises a strand displacement polymerase enzyme, and requires a circular amplification template, which may be provided by a circularised padlock probe. Amplification of the circular template provides a concatenated RCA product (RCP), comprising multiple copies of a sequence complementary to that of the amplification template (thus an RCP from a padlock probe comprises repeating units corresponding to the padlock probe). Such a concatemer typically forms a ball or “blob”, which may readily be visualised and detected.

Before the RCA reaction there may be an optional washing step. An RCA reaction is then typically initiated by adding one or more reagents for the RCA (termed “RCA reagents”). This will typically be the polymerase enzyme for RCA and nucleotides (specifically dNTPs), although optionally a primer for RCA may also be added. One or more of the RCA reagents may be added earlier.

The primer for the RCA reaction may be added to the reaction mixture, or may be pre-hybridised to the padlock probe. The binding site for the RCA primer may be provided in a region of the padlock probe which is different to the target binding regions (i.e. in the backbone region of the padlock). In some cases, the target nucleic acid molecule of the padlock probe (i.e. the molecule which serves as the ligation template for the padlock probe, e.g. the nucleic acid domain or blocking oligonucleotide) may serve as or provide the primer. Each of the padlock probes used in the present method may comprise a single common RCA primer binding site, so that the same primer can be used to prime RCA for all the nucleic acid products. Alternatively, a different primer may be used. The strand-displacing polymerase enzyme used for RCA is commonly Phi29 or a derivative thereof.

Alternatively, a PCR or other amplification reaction may be used to amplify part of the nucleic acid products. In particular, a PCR reaction may be used to amplify an identifier (e.g. barcode) sequence in each of the nucleic acid products. The nucleic acid products may each comprise shared primer binding sites flanking their barcode sequences, so that a single primer pair can be used to amplify the identifier sequences of each of the nucleic acid products.

The amplification products, or if there has been no subsequent amplification step, the nucleic acid products, are then detected. Any suitable detection means can be used for this purpose. Detection of the first amplification/nucleic acid product indicates the first target molecule (i.e. detection of the first amplification/nucleic acid product is a proxy for detection of the first target molecule), detection of the second amplification/nucleic acid product indicates the second target molecule (i.e. detection of the second amplification/nucleic acid product is a proxy for detection of the second target molecule), and detection of the third amplification/nucleic acid product indicates the indication between the first and second target molecule (i.e. detection of the second amplification/nucleic acid product is a proxy for detection of the interaction between the first and second target molecules).

Detection may involve the detection of a label incorporated into the product, e.g. means of using labelled nucleotides for synthesis of the product, or by attaching a label to the product in a subsequent step, for example by means of a labelled detection oligonucleotide (or detection probe) capable of hybridising specifically to the product, or it may comprise or involve sequencing of the product.

In the case of amplification by RCA, the amplification template (e.g. the circularised padlock probe) comprises a specific detection sequence (i.e. the identifier sequence), and thus the RCA product (RCP) (which as noted above is a concatemer of a circularised nucleic acid substrate, e.g. padlock probe) comprises repeating identifier sequences. Each of the first, second and third nucleic acid products comprises a unique identifier sequence. These products may be detected in any suitable manner. Typically, the identifier sequence provides a binding site for a detection oligonucleotide which hybridises to the identifier sequence. Detection oligonucleotides may analogously be used to bind to and detect other products, such HCR products or branched chain amplification products.

The detection oligonucleotide may carry a detectable label, also referred to as a detection moiety. The detection moiety is any moiety which can be detected, that is which can give rise, directly or indirectly, to a signal which can be detected. The detection moiety may thus be viewed as any detectable label, which may be directly or indirectly signal-giving. For example, the detection moiety may be spectroscopically or microscopically detectable, e.g. it may be a fluorescent or colorimetric label, a particle, e.g. a bead, or an enzymatic label. Any of the labels used in immunohistochemical techniques may be used. Hybridisation of the detection oligonucleotides to their multiple binding sites, concentrates them in the RCP, allowing it to be detected with high sensitivity.

The detection oligonucleotide need not, however, be directly labelled. For example, the detection oligonucleotide may be an unlabelled probe which functions as a sandwich probe. The concept of sandwich probes is well known in the art and may be applied according to any convenient protocol. The sandwich probes can bind to the RCP (or other product) but are not directly labelled themselves; instead, they comprise a sequence to which labelled secondary oligonucleotides can bind, thus forming a “sandwich” between the RCP and the labelled secondary oligonucleotide. Alternatively, the RCP may be detected indirectly, e.g. the product may be amplified by PCR and the amplification products may be detected.

The detection oligonucleotide or any secondary labelling probe may be labelled with a directly or indirectly detectable label. A directly detectable label is one that can be directly detected without the use of additional reagents, while an indirectly detectable label is one that is detectable by employing one or more additional reagents, e.g., where the label is a member of a signal producing system made up of two or more components. In many embodiments, the label is a directly detectable label, where directly detectable labels of interest include, but are not limited to: fluorescent labels, coloured labels, radioisotopic labels, chemiluminescent labels, and the like. In many embodiments, the label is a fluorescent label, where the labelling reagent employed in such embodiments is a fluorescently tagged nucleotide(s), e.g. fluorescently tagged CTP (such as Cy3-CTP, Cy5-CTP) etc. Fluorescent moieties which may be used to tag nucleotides for producing labelled probe nucleic acids (i.e. detection probes) include, but are not limited to: fluorescein, the cyanine dyes, such as Cy3, Cy5, Alexa 555, Bodipy 630/650, and the like. Other labels, such as those described above, may also be employed as are known in the art.

Conveniently, the detection moiety may be a coloured bead. Coloured beads may readily be visualised. Such beads, e.g. coloured polystyrene beads, are widely available.

Although various detection modalities may be employed, conveniently RCPs and other large multimeric products may be detected by visualisation, including by microscopy, or flow cytometry. In both cases directly or indirectly labelled detection oligonucleotides may be used, for example with fluorescent or coloured labels which may readily be detected. In this regard the label may include a bead or other detectable particle. In a microscopy-based method, RCPs or other multimeric products may be detected by imaging.

In other embodiments, the identifier sequence may be detected by quantitative PCR (qPCR) or sequencing. For instance, qPCR using “TaqMan” probes may be performed. In this instance, a probe complementary to each identifier sequence is used with each different probe being conjugated to a different, distinguishable fluorophore. Each identifier sequence (and thus each nucleic acid product) can thus be individually detected and quantified. When using qPCR for detection for example, circularised padlock probes may be used directly as the PCR template, or as noted above RCA may be performed and the RCP used as template.

Alternatively, when the identifier sequence is detected by sequencing, a form of high throughput DNA sequencing may be used. Sequencing by synthesis is a commonly used DNA sequencing method. Examples of sequencing by synthesis techniques include pyrosequencing, reversible dye terminator sequencing and ion torrent sequencing, any of which may be utilised in the present method. Conveniently, the identifier sequences are sequenced using massively parallel DNA sequencing. Massively parallel DNA sequencing may in particular be applied to sequencing by synthesis (e.g. reversible dye terminator sequencing, pyrosequencing or ion torrent sequencing, as mentioned above). Massively parallel DNA sequencing using the reversible dye terminator method may be performed, for instance, using an Illumina® NovaSeq™ system.

As is known in the art, massively parallel DNA sequencing is a technique in which multiple (e.g. thousands or millions or more) DNA strands are sequenced in parallel, i.e. at the same time. Massively parallel DNA sequencing requires target DNA molecules to be immobilised to a solid surface, e.g. to the surface of a flow cell or to a bead. Each immobilised DNA molecule is then individually sequenced. Generally, massively parallel DNA sequencing employing reversible dye terminator sequencing utilises a flow cell as the immobilisation surface, and massively parallel DNA sequencing employing pyrosequencing or ion torrent sequencing utilises a bead as the immobilisation surface.

As is known to the skilled person, immobilisation of DNA molecules to a surface in the context of massively parallel sequencing is generally achieved by the attachment of one or more sequencing adapters to the ends of the molecules. Thus, in the method herein, when the identifier sequence is amplified by PCR, the PCR primers may comprise adapters for sequencing (sequencing adapters) for addition to the identifier sequences, to enable sequencing of the products. Commonly, sequencing adapters are nucleic acid molecules (in particular DNA molecules). In this instance, short oligonucleotides complementary to the adapter sequences are conjugated to the immobilisation surface (e.g. the surface of the bead or flow cell) to enable annealing of the target DNA molecules to the surface, via the adapter sequences. Alternatively, any other pair of binding partners may be used to conjugate the target DNA molecule to the immobilisation surface, e.g. biotin and avidin/streptavidin. In this case biotin may be used as the sequencing adapter, and avidin or streptavidin conjugated to the immobilisation surface to bind the biotin sequencing adapter, or vice versa.

Sequencing adapters may thus be short oligonucleotides (preferably DNA), generally 10-30 nucleotides long (e.g. 15-25 or 20-25 nucleotides long). As detailed above, the purpose of a sequencing adapter is to enable annealing of the target DNA molecules to an immobilisation surface, and accordingly the nucleotide sequence of a nucleic acid adaptor is determined by the sequence of its binding partner conjugated to the immobilisation surface. Aside from this, there is no particular constraint on the nucleotide sequence of a nucleic acid sequencing adaptor.

In general, where amplification of the nucleic acid products is performed by RCA, the RCP is detected using a specific, labelled detection oligonucleotide. Such methods are particularly suited to in situ detection. Where amplification is performed by PCR, detection is generally performed by qPCR or sequencing. Such methods are particularly suited to non-in situ detection.

Thus, the nucleic acid/amplification products comprising the specific identifier sequences are detected. As set out above, when the first amplification/nucleic acid product is detected this indicates the presence of the first target molecule; when the second amplification/nucleic acid product is detected this indicates the presence of the second target molecule; when the third amplification/nucleic acid product is detected this indicates that complexes formed by interaction of the first and second target molecules are present. In embodiments in which a single padlock probe is used to detect a target molecule alone (when the target is not interacting with the other target molecule) or an interaction between the two molecules, the presence of the third amplification product but not the first or second indicates that the entirety of the target molecule in question is complexed with the other.

The method of the invention allows the detection of a pair of target molecules and their interaction, and in some embodiments the method may also allow their quantification. The quantification may be relative, i.e. the relative amounts of the two target molecules in the sample may be quantified, along with the proportion of each which are interacting. Alternatively, the quantification may be absolute, whereby the concentrations of the two target molecules in the sample, and of the interaction complex containing them, are calculated.

Relative quantification can be accomplished by comparison of detected levels or amounts between two or more different target molecules, to provide a relative quantification of each of the two or more different nucleic acid molecules or sequences, i.e., relative to each other. Thus, ratios of target molecules present in a sample may be determined. Absolute quantification generally requires generation of a standard curve using known concentrations of the target molecules against which the detection values can be compared.

In a particular embodiment, the nucleic acid products (circularised substrates) are amplified by RCA. The RCPs are detected and the amounts of the respective targets quantified by RCA signal counting. Since RCPs may be individually detected, RCA and RCP detection provides a readily quantifiable method, by means of which the detected molecule or interaction may be quantified, by counting or enumerating the number or amount of RCPs. Such quantification of RCPs is well known in the art and has been widely described in the literature.

Alternatively, quantification of the targets detected by qPCR is routine in the art, including absolute quantification qPCR using a standard curve. Levels of the target molecules can also be quantified during sequencing, if sequencing is used for detection of the amplification products. The level of amplification product generated should be proportionate to the amount of target present in the sample. Use of a common primer binding site for amplification helps to ensure that the levels of different amplification products are comparable and proportionate to their concentrations. The levels of each amplification product sequenced are detected during sequencing allowing their relative concentrations to be calculated. Again, generation of a standard curve using known concentrations of each target molecule, against which the experimental values can be compared, allows absolute quantification of the target molecule concentrations.

The present method may be performed in multiplex, to detect multiple different pairs of target molecules and their interactions. To multiplex the method a number of options are available. Clearly the proximity probes for each individual reaction in a multiplex reaction will comprise different binding domains, specific for the target molecules of interest. In one embodiment, the nucleic acid domains are the same in all proximity probe pairs. In this embodiment, substrate nucleic acid molecules (particularly padlock probes) are pre-hybridised to the proximity probes, before they are applied to the sample. Different padlock probes, e.g. carrying different identifier sequences so they can be distinguished, are pre-hybridised to each proximity probe pair. The method is then performed as described above. FIG. 4 illustrates a multiplex embodiment in which different padlock probes are used for the detection of different target molecules and their interactions. Here, padlocks A, B and C are used for the detection of target molecules X and Y and their interaction, and padlocks D, E, and F are used to detect target molecules X1 and Y1, and their interaction. The same nucleic acid domains are conjugated to the different binding domains (here represented by antibodies) used to detect the different targets X and X1, and Y and Y1 respectively.

In another embodiment, different nucleic acid domains are used in each proximity probe pair. These may be used with different padlock probes specific for each pair of nucleic acid domains. However, in in a still further embodiment a single set of padlock probes is used for all of the multiplex reactions. In this embodiment, the padlock probes used are gap-fill padlock probes. Each nucleic acid domain comprises a padlock binding site comprising a unique identifier sequence flanked by sequences complementary to the target-binding sequences of the relevant padlock probe. Upon padlock probe binding to a nucleic acid domain, a gap-filling reaction is performed followed by ligation of the padlock probe, incorporating the identifier sequence from the nucleic acid domain into the padlock probe, for subsequent identification. The gap-filling may be by extension or by a gap oligonucleotide complementary to the gap sequence (which comprises or constitutes the identifier sequence). Such an embodiment is depicted in FIG. 5.

In this way, the method may be used to detect multiple pairs of target molecules in a sample, and their interaction. The term “multiple” as used herein means two or more, for example, 3, 4, 5, 6, 8, 9, 10, 15, 20, 25, 30, 40, 50, 60, 70, 80, 90, or 100 or more.

When the method is run in multiplex, detection of the amplification product may be performed by sequencing, as described above. In this way, an essentially limitless number of different sequences can be detected in any one go. This may be advantageous for very high multiplex methods. Alternatively, different or distinguishable labels may be used, although the level of multiplex may be limited by the number of available labels. To increase the level of multiplexing, combinatorial or sequential labelling methods may be used, according to principles well known in the art. It is known for example, to decode barcode sequences using sequencing-by-hybridisation methods which involve the use of labelled hybridization probes in sequence.

A particular embodiment of the present method is shown in FIG. 1. This embodiment provides a method for detecting two target molecules (X and Y) in a sample, and detecting an interaction between the two target molecules, the method comprising:
(i) contacting the sample with:
   (a) a first proximity probe (A probe), for the first target molecule, wherein the first proximity probe comprises a first nucleic acid domain comprising one or more single-stranded regions (a single strand is shown), wherein the first nucleic acid domain comprises a padlock binding site and a second sequence for hybridisation of a padlock (a padlock hybridisation sequence), both being located within the one or more single-stranded regions;
   (b) a second proximity probe (B probe), for the second target molecule, wherein the second proximity probe comprises a second nucleic acid domain comprising one or more single-stranded regions (a single strand is shown), wherein the second nucleic acid domain comprises a padlock binding site and a further padlock hybridisation sequence, both being located within the one or more single-stranded regions;
   (c) a first padlock probe (A) which comprises at its 5′ and 3′ ends binding sequences capable of hybridising to the padlock binding site of the first proximity probe;
   (d) a second padlock probe (B) which comprises at its 5′ and 3′ ends binding sequences capable of hybridising to the padlock binding site of the second proximity probe; and
   (e) a third padlock probe comprising one or two circularisation oligonucleotides (C, or C1 and C2), wherein:
      (I) the one circularisation oligonucleotide (C) comprises target-binding sequences at its 5′ and 3′ ends capable of hybridising to the hybridisation sequence of the second proximity probe, and between the 5′ and 3′ ends an anchor sequence capable of hybridising to the hybridisation sequence of the first proximity probe, and wherein when applied to the sample the circularisation oligonucleotide is pre-hybridised at the anchor sequence to the hybridisation sequence of the first proximity probe; or
      (II) the two circularisation oligonucleotides (C1 and C2) together form a two-part padlock probe, each circularisation oligonucleotide comprising at its 5′ and 3′ ends a first target-binding sequence capable of hybridising to the hybridisation sequence of the first proximity probe and a second target binding sequence capable of hybridising to the hybridisation sequence of the second proximity probe, such that when the first and second proximity probes are in proximity both circularisation oligonucleotides hybridise to both proximity probes and their respective ends are brought into juxtaposition for ligation, directly or indirectly, to each other to form a circle
(ii) where the 5′ and 3′ ends of the padlock probes have hybridised to their respective binding sites or hybridisation sequences with a gap between them, performing a gap-filling reaction, and ligating the hybridised padlock probes and one or two circularisation oligonucleotides, thereby generating a first circular nucleic acid product from the first padlock probe, a second circular nucleic acid product from the second padlock probe, and a third circular nucleic acid product from the third padlock probe, wherein the first, second and third circular nucleic acid products are distinguishable from one another by sequence (e.g. each comprise a distinguishable identifier sequence);
(iii) amplifying the first, second and third circular nucleic acid products to generate a first, second and third amplification product; and
(iv) detecting the first, second and third amplification product, wherein the first amplification product indicates the first target molecule, the second amplification product indicates the second target molecule, and the third amplification product indicates the interaction between the two target molecules.

The figure exemplifies a particular design of proximity probe, but any suitable design may be used (in this and all embodiments). For instance, the figure shows the padlock binding sites to be located at the end of the nucleic acid domain distal to the binding domain, and the hybridisation sequences to be proximal to the binding domain. However, this arrangement is not required: any functional arrangement of binding sites/hybridisation sequences can be used. Similarly, any design in respect of single-and double-stranded regions of the probes may be used, so long as both binding site and hybridisation sequences on each probe are located within single-stranded regions. The figure shows two probe designs in this respect: the A probe is entirely single-stranded, whereas the B probe has a double-stranded central region with both strands having single-stranded overhangs comprising the binding site and hybridisation sequences (see FIG. 1A or 1B). Such a set-up may be used, but the probes used may alternatively all have the same structure (e.g. that of the A probe or that of the B probe). FIG. 1C shows both nucleic acid domains, one on each probe, to be single-stranded. Entirely different probe structures may also be used, e.g. the nucleic acid may have a double-stranded region at one end (distal or proximal to the binding domain) with a single-stranded overhand at only one end, comprising both hybridisation sequences.

As a variant of the format shown in FIG. 1A, each of the 3 padlock probes may be provided with a blocking oligonucleotide, as described above.

A further variant is shown in FIG. 8, which shows an embodiment in which both nucleic acid domains of the proximity probes are partially double-stranded. In particular, each nucleic acid domain has a double-stranded central region with both strands having single-stranded overhangs comprising the binding site and hybridisation sequences. This configuration is shown in the Examples below to work well. In such an embodiment, one or both strands of the nucleic acid domain to which the third padlock probe binds (and which templates its ligation) may further act a primer for RCA of the ligated and circularised third padlock probe.

The term “hybridisation sequence” as used here refers to the sequence to which the padlock probe corresponding to the third nucleic acid substrate molecule binds. As noted above, in this embodiment the third nucleic acid substrate molecule is a one-or two-part padlock probe.

Another embodiment is shown in FIG. 2. This embodiment provides a method for detecting two target molecules (X and Y) in a sample, and detecting an interaction between the two target molecules, the method comprising:
(i) contacting the sample with:
   (a) a first proximity probe (A probe) for the first target molecule, wherein the first proximity probe comprises a nucleic acid domain (here depicted as a single-stranded nucleic acid domain) hybridised to a first padlock probe, wherein the 5′ and 3′ ends of the first padlock probe are hybridised to a blocking oligonucleotide;
   (b) a second proximity probe (B probe) for the second target molecule, wherein the second proximity probe comprises a nucleic acid domain comprising one or more single-stranded regions, wherein the nucleic acid domain comprises a first padlock binding site capable of hybridising to the 5′ and 3′ ends of the first padlock probe, and a second padlock binding site for a second padlock probe, the padlock binding sites both being located within the one or more single-stranded regions; and
   (c) a second padlock probe (B), which comprises at its 5′ and 3′ ends target-binding sequences capable of hybridising to the second padlock binding site of the second proximity probe;
   such that when the first and second proximity probe are in proximity the blocking oligonucleotide is displaced from the first padlock probe by the first padlock binding site of the second proximity probe, wherein the blocking oligonucleotide and/or the first padlock binding site of the second proximity probe comprise a gap sequence located between the sequences capable of hybridising to the 5′ and 3′ ends of the first padlock probe, such that the hybridised 3′ and 5′ ends of the first padlock probe are separated by a gap;
(ii) where the 5′ and 3′ ends of the padlock probes have hybridised to their respective binding sites with a gap between them, performing a gap-filling reaction, and ligating the hybridised padlock probes, thereby generating a first circular nucleic acid product from the first padlock probe hybridised to the blocking oligonucleotide, a second circular nucleic acid product from the second padlock probe, and a third circular nucleic acid product from the first padlock probe hybridised to the first padlock binding site of the second proximity probe, wherein the first, second and third circular nucleic acid products are distinguishable from one another by sequence (e.g. each comprise a distinguishable identifier sequence);
(iii) amplifying the first, second and third circular nucleic acid products to generate a first, second and third amplification product; and
(iv) detecting the first, second and third amplification products (e.g. by detecting their identifier sequences), wherein the first amplification product indicates the first target molecule not interacting with the second target molecule, the second amplification product indicates the second target molecule, and the third amplification product indicates the interaction between the two target molecules.

Another embodiment of the invention is shown in FIG. 3A. This embodiment provides a method for detecting two target molecules (X and Y) in a sample, and detecting an interaction between the two target molecules, the method comprising:
(i) contacting the sample with:
   (a) a first proximity probe pair comprising a first and second proximity probe (x1 and x2 probes) for the first target molecule, each proximity probe comprising a nucleic acid domain comprising one or more single-stranded regions, wherein the nucleic acid domain of the first proximity probe comprises a first padlock binding site for a first padlock probe and the nucleic acid domain of the second proximity probe comprises a hybridisation sequence capable of hybridising to a third padlock probe, the first padlock binding site and the hybridisation sequence being located within the one or more single-stranded regions;
   (b) a second proximity probe pair comprising a first and second proximity probe (y1 and y2 probes) for the second target molecule, each proximity probe comprising a nucleic acid domain comprising one or more single-stranded regions, wherein the nucleic acid domain of the first proximity probe comprises a second padlock binding site for a second padlock probe and the nucleic acid domain of the second proximity probe comprises a hybridisation sequence capable of hybridising to a third padlock probe, the second padlock binding site and the hybridisation sequence both being located within the one or more single-stranded regions;
   (c) a first padlock probe (A) which comprises at its 5′ and 3′ ends target-binding sequences capable of hybridising to the first padlock binding site of the first proximity probe of the first proximity probe pair;
   (d) a second padlock probe (B) which comprises at its 5′ and 3′ ends target-binding sequences capable of hybridising to the second padlock binding site of the first proximity probe of the second proximity probe pair;
   (e) a third padlock probe which comprises one or two circularisation oligonucleotides, wherein:
      (I) the one circularisation oligonucleotide (C) comprises target-binding sequences at its 5′ and 3′ ends capable of hybridising to the hybridisation sequence of the second proximity probe of the second proximity probe pair and an anchor sequence capable of hybridising to the hybridisation sequence of the second proximity probe of the first proximity probe pair, wherein when applied to the sample the anchor sequence is hybridised to the hybridisation sequence of the second proximity probe of the first proximity probe pair; or
      (II) the two circularisation oligonucleotides together form a two-part padlock probe, each circularisation oligonucleotide comprising at its 5′ and 3′ ends a first target-binding sequence capable of hybridising to the hybridisation sequence of the second proximity probe of the first proximity probe pair and a second target-binding sequence capable of hybridising to the hybridisation sequence of the second proximity probe of the second proximity probe pair, such that when the first and second proximity probes are in proximity, each circularisation oligonucleotide hybridises to the nucleic acid domains of both second proximity probes, and the respective 5′and 3′ends of the two circularisation oligonucleotides are brought into juxtaposition for ligation, directly or indirectly, to each other to form a circle;
(ii) where the 5′ and 3′ ends of the padlock probes have hybridised to their respective binding sites or hybridisation sequences with a gap between them, performing a gap-filling reaction, and ligating the hybridised padlock probes, thereby generating a first circular nucleic acid product from the first padlock probe, a second circular nucleic acid product from the second padlock probe, and a third circular nucleic acid product from the third padlock probe, wherein the first, second and third circular nucleic acid products distinguishable from one another by sequence (e.g. each comprise a distinguishable identifier sequence);
(iii) amplifying the first, second and third circular nucleic acid products to generate a first, second and third amplification product; and
(iv) detecting the first, second and third amplification products (e.g. by detecting their identifier sequences), wherein the first amplification product indicates the first target molecule, the second amplification product indicates the second target molecule, and the third amplification product indicates the interaction between the two target molecules.

As exemplified in FIG. 3A, in embodiments in which pairs of proximity probes are used to detect the first and second target molecules, the first and second padlock probes may be capable of hybridising to both members of the first proximity probe pair and second proximity probe pair, respectively. When this is the case, the padlock probe hybridises to one member of the probe pair via the target-binding sites of the padlock probe, and to the other member of the probe pair via a sequence in the padlock probe backbone (i.e. an anchor sequence), as shown in FIG. 3A. In this instance the padlock probe is preferably pre-hybridised to the second member of the proximity probe pair (i.e. the proximity probe to which the padlock probe binds via an anchor sequence) before the probes are applied to the sample, in order for binding of both proximity probes to the target to be required for padlock probe ligation and amplification. A similar arrangement is shown in FIG. 13. However, in another embodiment, as shown in FIG. 6, the first and second padlocks hybridise to only one of the proximity probes of a proximity probe pair, and in particular to a different probe of the two proximity probe pairs, than does the third padlock probe.

In other words, the padlock probe may bind via its target-binding sequences to the proximity probe which comprises the hybridisation sequence, or to the other proximity probe (i.e. the proximity probe which does not comprise the circularisation sequence.

Thus, in the configurations shown in FIGS. 3A, 6 and 13, the third proximity probe pair is made up of the second proximity probe from the first and second proximity probe pairs. The hybridisation site in the nucleic acid domain of each said second proximity probe which is capable of hybridising to the third padlock probe thus constitutes a separate region of complementarity in the nucleic acid domain of the proximity probes which is capable of mediating the interaction between the nucleic acid domains of the third proximity probe pair. As indicated above, this additional region of complementarity may be in the nucleic acid domain of the same proximity probe to which the first or second padlock hybridises (as shown in FIG. 3A, or in the first proximity probe pair of FIG. 13), or it may be on the other proximity probe of the proximity probe pair (as shown in FIG. 6).

As mentioned above, the configurations shown in FIGS. 3A, 6 and 13 may be modified to incorporate blocking oligonucleotides on the three padlocks.

As described above, in a further embodiment, the format of FIG. 6 may be modified to replace the proximity probe pairs used to detect the individual target molecules with a single probe which carries two separate nucleic acid domains, as depicted in FIG. 7. The two single probes used to detect A and B together make up a proximity probe pair to detect the AB interaction,

Another embodiment is shown in FIG. 3B. This embodiment provides a method for detecting two target molecules in a sample (X and Y), and detecting an interaction between the two target molecules, the method comprising:
(i) contacting the sample with:
   (a) a first proximity probe pair comprising a first and second proximity probe for the first target molecule, wherein the first proximity probe comprises a nucleic acid domain (here depicted as a single-stranded nucleic acid domain) comprising a first padlock binding site(Template a), and the second proximity probe comprises a single-stranded nucleic acid domain hybridised to a first padlock probe (Padlock a) which comprises at its 5′ and 3′ ends binding sequences capable of hybridising to the first padlock binding site; and
   (b) a second proximity probe pair comprising a first and second proximity probe for the second target molecule, wherein the first proximity probe comprises a single-stranded nucleic acid domain comprising a second padlock binding site (Template b), and the second proximity probe comprises a single-stranded nucleic acid domain hybridised to a second padlock probe (Padlock b) which comprises at its 5′ and 3′ ends binding sequences capable of hybridising to the second padlock binding site;
wherein the binding sequences of the first padlock probe are capable of hybridising to the second padlock binding site, and/or the binding sequences of the second padlock probe are capable of hybridising to the first padlock site;
wherein the first and second padlock probes each comprise an identifier sequence, and the first and/or second padlock binding sites comprise a gap sequence located between the sequences capable of hybridising to the 5′ and 3′ ends of the respective padlock probes;
(ii) where the 5′ and 3′ ends of a padlock probe have hybridised to their respective binding sites with a gap between them, performing a gap-filling reaction, and ligating the hybridised padlock probes, thereby generating a first circular nucleic acid product from the first padlock probe hybridised to the first padlock binding site, a second circular nucleic acid product from the second padlock probe hybridised to the second padlock hybridisation binding site, and a third and, optionally, fourth circular nucleic acid product from the first padlock probe hybridised to the second padlock binding site and/or the second padlock probe hybridised to the first padlock binding site, wherein the first, second, third and optionally fourth circular nucleic acid products are distinguishable from one another by sequence (e.g. each comprise a unique combination of identifier sequence and optional gap sequence);
(iii) amplifying the first, second, third and optional fourth circular nucleic acid products to generate a first, second, third and, optionally, fourth, amplification product; and
(iv) detecting the first, second, third and optional fourth amplification products (e.g. by detecting their unique combination of identifier and optional gap sequences), wherein the first amplification product indicates the first target molecule, the second amplification product indicates the second target molecule, and the third amplification product, and optional fourth amplification product, indicate the interaction between the two target molecules.

As set out above, in this embodiment, the first padlock probe is pre-hybridised to the second proximity probe of the first proximity probe pair and the second padlock probe is pre-hybridised to the second proximity probe of the second proximity probe pair, i.e. both padlock probes are hybridised to their respective proximity probes before the proximity probes are applied to the sample. As shown in FIG. 3B, the padlock probes are pre-hybridised to their respective proximity probes via sequences in their backbones (i.e. anchor sequences).

The method may be a homogenous method, that is a method performed in solution, or it may be a heterogenous method, that is a method performed on or using a solid phase. This may depend on the sample which is used, and/or the target molecule or analyte it is desired to detect. For example, the method may be performed on cell or tissue samples for in situ detection. This is desirable in instances of localised detection. Thus, the method may be used to determine the spatial distribution of the target molecules, and the interaction. Alternatively, for other samples or detection assays, the method may be performed in solution, for example with liquid samples, e.g. plasma or serum samples etc., or processed samples etc.

A second aspect herein concerns a kit for performing the methods detailed above. The kit comprises the necessary components to carry out the methods. The kit may provide components for any experimental set up. In particular, the kit comprises a first proximity probe or proximity probe pair for detection of a first target molecule, and a second proximity probe or proximity probe pair for detection of a second target molecule, wherein said probes each comprise a binding domain capable of hybridising directly or indirectly to their target molecule and a nucleic acid domain, and wherein the first and second probe, or one of the probes of the first proximity probe pair and one of the probes of the second proximity probe pair, together form a third proximity probe pair for detection of the interaction between the first and second target molecules.

The kit also comprises at least first and second nucleic acid reagents as defined and described above. In an embodiment, the kit comprises first and second nucleic acid substrate molecules, wherein the first substrate molecule is capable of hybridising to the nucleic acid domain of the first probe or one of the probes of the first proximity probe pair and the second substrate molecule is capable of hybridising to the nucleic acid domain of the second probe or one of the probes of the second proximity probe pair. As detailed above, it is preferred that the nucleic acid substrate molecules are padlock probes.

In an embodiment, the kit additionally comprises a third nucleic acid substrate molecule, particularly a third padlock probe. The third nucleic acid substrate molecule is capable of hybridising to the reporter nucleic acid domains of both members of the third proximity probe pair. As detailed above, the third nucleic acid substrate molecule is preferably a padlock probe, which may be in one or two parts.

Depending on the experimental set up, the first, second and/or third nucleic acid substrate molecules may be provided in the kit pre-hybridised to their respective proximity probes, as described above in respect of the methods.

The kit may further comprise enzymes for use in the methods. The kit may comprise a ligase as detailed above for ligation of padlock probes. When using gap-fill padlock probes, the kit may comprise a polymerase for use in the extension reaction to fill the gap. Alternatively, when using one or more gap-fill padlock probes the kit may comprise one or more gap oligonucleotides capable of filling in the gap by hybridising to the gap sequence.

The kit may also comprise reagents for use in amplification and detection. For instance, the kit may comprise a strand-displacing nucleic acid polymerase for use in RCA, preferably phi29 polymerase. The kit may also or alternatively comprise components for use in qPCR amplification of the padlock probe detection sequences. The kit may also comprise detection oligonucleotides suitable for detecting each of the nucleic acid products or their amplicons.

When amplification product detection is to be performed by sequencing, components for massively parallel DNA sequencing may also be provided. For instance, the kit may comprise components for PCR amplification of the DNA identifier/barcode sequences to be sequenced, including primers with 3′ sequencing adapter sequences, a polymerase, nucleotides, etc. The product may comprise a solid base to which the amplification products can be immobilised for sequencing, e.g. a flow cell or bead.

As indicated above, the methods and kits herein may have many different uses and applications, including in any detection assay where it is desired to detect or characterise an interaction. This may include, for example, detecting target analytes, including for diagnostic use or for study of interactions. They may be used for profiling or characterising interactions. The methods and kits may find particular application in the development of bi-specific binding molecules, such as bi-specific antibodies, which comprise two different binding domains, each specific for (i.e. targeting) a different antigen or molecule. In the design of such molecules, it may be desirable to profile in situ the two target molecules intended for the bi-specific binding molecule to determine their individual cellular expression and their cellular expression in proximity, and the methods herein are particularly suitable for such use. Further, bi-specific binding molecules can be used to bring the two targeted molecules, e.g. two proteins, into closer proximity. The methods and kits herein could be used to determine if such an effect occurs.

The methods herein may be further understood by reference to the figures and non-limiting examples below.

### DESCRIPTION OF FIGURES

FIG. 1 depicts schematics for assay formats using a single pair of proximity probes (comprising A probe and B probe) to detect both two target molecules, X and Y, individually, and the interaction between them. Padlock probes A and B are used to detect X and Y respectively. Padlock probe C is used to detect the interaction. (A) shows a 1-part padlock probe C which is pre-hybridised in its backbone region to the nucleic acid domain of the A probe; (B) shows a two-part padlock probe C comprising c1 and c2, where each of c1 and c2 hybridise to the nucleic acid domains of both A probe and B probe when they are brought into proximity (when bound to X and Y in an interaction); (C) shows a configuration in which each of the nucleic acid domains of the A probe and the B probe are single-stranded and a 2-part padlock probe C is used—in this configuration a separate primer is used for RCA.

FIG. 2 depicts a schematic for an assay format using a single pair of proximity probes (comprising A probe and B probe) and 2 padlock probes to detect both two target molecules, X and Y, individually, and the interaction between them. The A probe hybridises to the backbone of a first padlock probe, the target-binding ends of which are prehybridised, with a gap between them, to a blocking oligonucleotide. The B probe hybridises to the target-binding ends of a second padlock probe, padlock B. When X and Y are in an interaction, the A and B probes are brought into proximity, and the first padlock is able to hybridise to its target binding site on the B probe (the blocking oligonucleotide is displaced), which templates the ligation of the padlock probe, allowing the detection of the X-Y interaction. Padlock B resolves the level of target molecule Y. When X and Y are not interacting, the A and B probes are not in proximity, and the target molecule X may be detected using the first padlock and a gap-fill reaction using the blocking oligonucleotide.

FIG. 3 depicts schematics for assay formats using two pairs of proximity probes, where probe pair x1 and x2 are used to detect target molecule X via dual recognition and probe pair y1 and y2 are used to detect target molecule Y via dual recognition and where x1 and y1 together form a third proximity probe pair for detection of the X-Y interaction. (A) shows three separate padlocks A, B and C, used respectively to detect X, Y and the X-Y interaction; (B) shows 2 padlocks, padlocks a and b respectively, and two ligation templates for the padlocks, templates a and b respectively (the nucleic acid domains of one of each pair of proximity probes). To detect X, padlock a is ligated on template a, to detect Y, padlock a is ligated on template b, and to detect the X-Y interaction, padlock b is ligated on template a and or padlock a is ligated on template b. Padlocks a and b comprise identifier sequences by which they may be distinguished, and a gap-fill reaction allows the template used for ligation of the padlocks to be determined.

FIG. 4 depicts a multiplex assay format, in which proximity probe pairs (A, B) and (D, F) for detection of different target molecule combinations (X, Y) and (X1, Y1) are used in combination with different padlock probes, (A, B, C) and (D, E, F) respectively. The nucleic acid domains used in the respective proximity probe pairs may be the same or different.

FIG. 5 depicts an alternative multiplex assay format, wherein multiplexing is achieved by gap-filling. A standard set of gap-fill padlock probes A, B, and C is used, in combination with multiple proximity probe pairs for detection of different target molecule combinations, the nucleic acid domains of which comprise gap sequences which may be used to distinguish the circularised padlock probes. Gap-fill may be by gap-fill extension using the gap sequence as template, or by hybridising complementary gap oligonucleotides to the gap sequences.

FIG. 6 depicts a schematic for another assay format using two pairs of proximity probes which act as secondary reagents which bind to a primary binding partner capable of binding to the target molecule, to detect first and second target molecules A and B, and the interaction between them (AB). The first proximity probe pair comprises a first probe which binds to the primary binding partner (depicted as an antibody) for the first target molecule, and the nucleic acid domain of the first probe of the first proximity probe pair is detected by a first padlock probe to detect the first target molecule (A). The second proximity probe pair comprises a first probe which binds to the primary binding partner (depicted as an antibody) for the second target molecule, and the nucleic acid domain of the first probe of the second proximity probe pair is detected by a second padlock probe to detect the second target molecule (B). The first and second proximity probe pairs each comprise a second probe which second probes together constitute a secondary reagent (“third proximity probe pair”) used to detect the interaction between target molecule A and target molecule B. A third padlock probe, provided in 2 parts, is used to detect the third proximity probe pair when both the second probes have bound to their respective targets (primary binding partners) in proximity, which occurs when both target molecules A and B are in an interaction (AB). One end of each of the two parts of the third padlock probe hybridises to one of the nucleic acid domains of the second probes which constitute the third proximity probe pair, allowing the ends of the respective parts of the third padlock probe to be ligated together.

FIG. 7 depicts a modification of the assay format of shown in FIG. 6, in which rather than first and second proximity probe pairs, two single probes (first and second probes are used, and both probes together make up the third proximity probe pair. The first and second probes each carry two separate nucleic acid domains, the first nucleic acid domain of each probe being detected by first and second padlock probes respectively, to detect the individual target molecule A or B. The second nucleic acid domain of each probe is capable of hybridising to the one end of each part of a 2-part third padlock probe, to allow an interaction between A and B (AB) to be detected using the third padlock probe.

FIG. 8 depicts a schematic for a modification of the embodiments shown in FIG. 1A-C, in which the nucleic acid domains of both probes of a single pair of proximity probe used to detect both two target molecules, A and B, individually, and the interaction between them (AB), are double-stranded. Padlock probes A and B are used to detect target molecules A and B respectively, by binding to the first strand of the nucleic acid domains of the first and second probes of the proximity probe pair respectively. A third padlock probe, provided in 2 parts, is used to detect the interaction, where each of the two parts of the third padlock probe hybridise to the second strand of the nucleic acid domains of both the first and second probe when they are brought into proximity (when bound to target molecules A and B in an interaction). In this configuration a separate primer is not needed for the RCA, as the second strand of the nucleic acid domain(s), which acts as target (i.e., ligation template) for the padlock probe is able to prime the RCA reaction.

FIG. 9 shows the results of using the method to detect beta-catenin, E-cadherin, and the interaction between the proteins on MCF7 cells. The panel of images is divided along rows based on the antibodies present and along columns based on the fluorophore imaged. The top row shows the signal generated from all the different assay reactions with a signal generated in three fluorophore channels, indicating the presence of the two individual proteins and the protein interaction. The second row displays the technical control where only the anti-beta catenin antibody is present, which in turn means that the only channel that generates a positive signal is the FITC channel corresponding to anti-beta catenin. Similarly, in row three, only signal was generated in the Texas red channel (TxR) corresponding to the antibody present and leaving the interaction and Beta-catenin reaction blank. The bottom row with no primary antibodies is not generating any signal.

FIG. 10 shows the detection of beta-catenin, E-cadherin, and the interaction between the proteins on FFPE skin tissue slides. The panel of images is divided along rows based on the fluorophore imaged and along columns based on the antibodies present. Top row show cases that both antibodies are needed for the assay reactions to generate the signal indicating an interaction (image furthest to the right). While in rows two and three, the signal is generated in all the conditions where the assay reaction target is present, resulting in a signal in two of the images per row.

FIG. 11 shows the detection of PDGF Receptor-beta phosphorylation (pPDGFR-beta) on BJ-hTert cells. The BJ-hTert cells were starved in serum-free growth media overnight and were after that stimulated with PDGFbb for 45 min on ice or left as a control. The proximity of an anti-PDGFR beta antibody and an anti-pan phosphor-tyrosine (pan-P-Tyr) antibody allows for an assay reaction that result in the detection of PDGFR-beta phosphorylation. Simultaneously the other two assay reactions allow for the analysis of total PDGFR-beta and total pan-P-Tyr in the sample. In the top row, there is an increased amount of signal for the total phosphorylation and phosphorylated receptor detection after stimulation compared to the staved cells from the bottom row. The total receptor signal is also slightly downregulated in the stimulated cells.

FIG. 12 shows results validating the format of the method depicted in FIG. 1A, wherein the proximity probes are either secondary (A) or primary (B) conjugated antibodies. In (A) the proximity probes pair is used to detect E-cadherin and beta-catenin, located at the cell membrane, and their interaction. In (B) the proximity probe pair is used to detect Lamin B1 and Lamin A proteins, mainly located in the nuclei, and their interaction.

FIG. 13 depicts a schematic for an assay format using two pairs of proximity probes, where a first proximity probe pair is used to detect a first target molecule via dual recognition, and a second proximity probe pair is used to detect a second target molecule via dual recognition, and where the second proximity probe member of the first and second proximity probe pair together form a third proximity probe pair for detection of the interaction between the target molecule. Three separate padlocks are used respectively to detect the first and second target molecules and the interaction.

FIG. 14 shows the results of the detection of beta-catenin, E-cadherin, and the interaction between the proteins in FFPE tissue slides, using the method format depicted in FIG. 6, when performed on a slide autostainer. The panel of images is divided along rows based on the fluorophore imaged and along columns based on the antibodies present.

FIG. 15 shows the detection of (A) molecule A (COX1) with FITC label, (B) molecule (B) GM130 with Cy3 label, and (C) molecules A and B COX1/GM130 with Cy5 label, using a detection method with the format depicted in FIG. 6. In all three images DAPI staining is also shown. Molecules A and B do not interact, and as expected no interaction/proximity signal is detected in (C).

### EXAMPLES

### Example 1

### Detection of Beta-Catenin, E-Cadherin, and the Interaction Between the Proteins on MCF7 Cells.

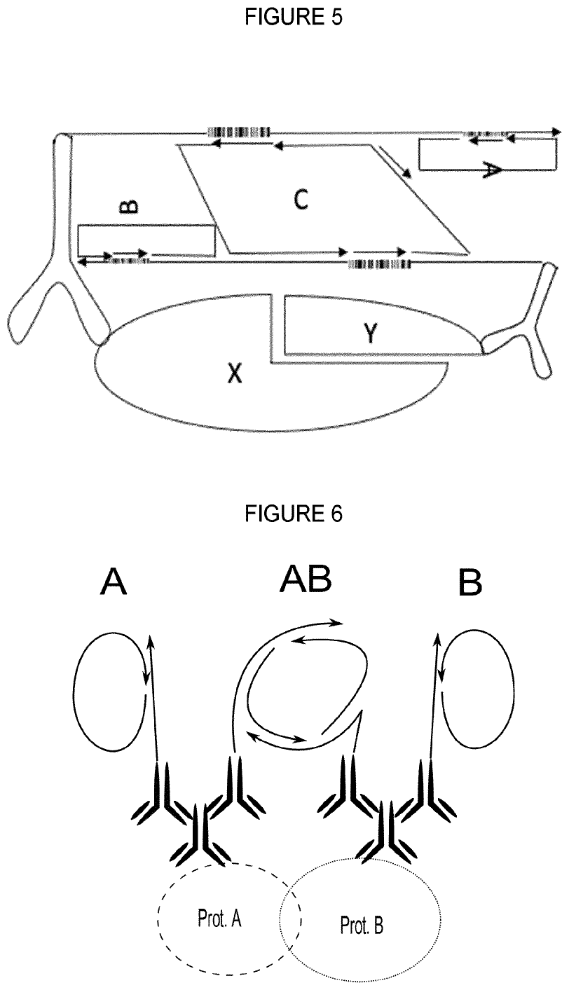

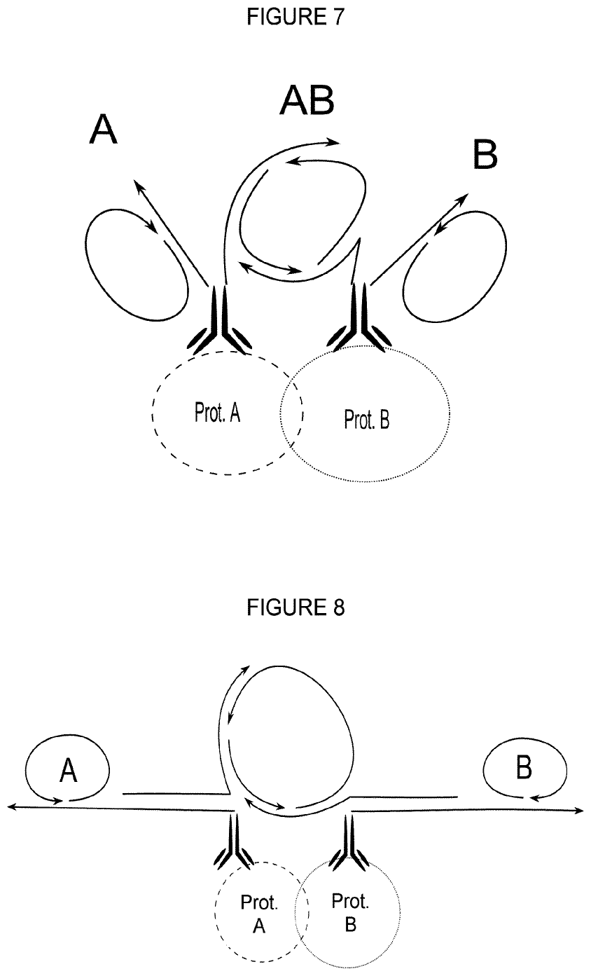

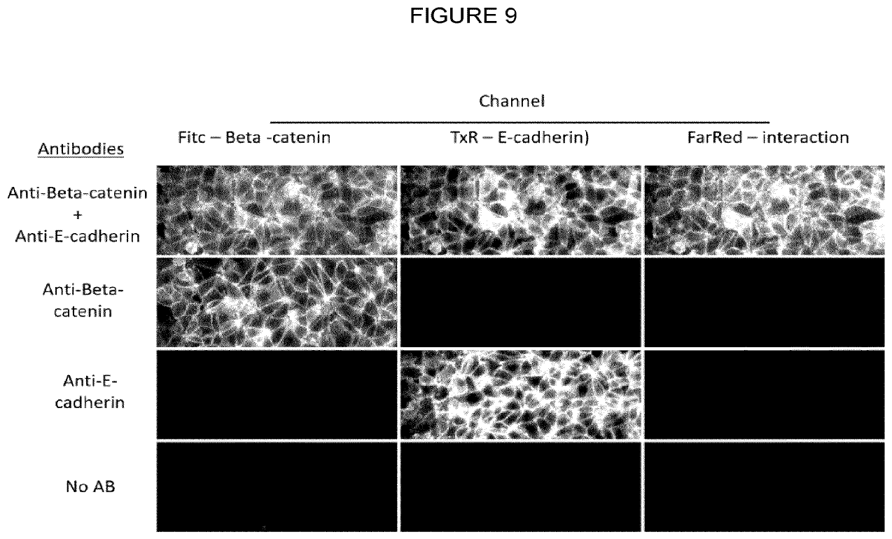

An experiment was performed to demonstrate the performance of the method having the format depicted in FIG. 6. MCF7 cells were grown to a high density to ensure tight cell-to-cell contact and the formation of a complex between beta-catenin and E-cadherin. Cells were incubated with a blocking reagent to minimize nonspecific staining. The proteins beta-catenin (target molecule A) and E-cadherin (target molecule B) were detected in MCF7 cells by incubating with primary antibodies, rabbit anti-beta-catenin, and mouse anti-E-cadherin. Absence of primary antibodies was used as negative control. Subsequently, the cells were contacted with proximity probes pairs A (anti-rabbit) and B (anti-mouse). Three padlock probes were used to detect respectively, beta-catenin (A), E-cadherin (B) and their interaction (AB). After hybridisation to their respective targets (nucleic acid domains of the proximity probes), the padlocks were circularised by ligation, and subjected to RCA to generate RCA products, which were detected by using fluorescently labelled detection probes. The first probe of pair A was detected by a first padlock probe (A) to detect beta-catenin (FITC channel), the first probe of pair B was detected by a second padlock probe (B) to detect E-cadherin (Texas Red (TxR) channel). The second probes of pairs A and B were detected by a third padlock probe to detect the interaction (AB) (FarRed channel). The results are shown in FIG. 9.

The top row shows the signals generated from all three padlocks, using proximity probe pairs A and B. The presence of the individual proteins (columns 1 and 2) and of the interaction (column 3) can be detected.

The second row shows the signals generated where only the anti-beta-catenin primary antibody is used (omission of the anti-E cadherin primary antibody). In this case, signal is generated only from the first padlock A (column 1, FITC channel) corresponding to beta-catenin; no signal is generated for E-cadherin or for the interaction.

Conversely, the third row shows that where only the anti-E-cadherin primary antibody is used (omission of the anti-beta-catenin antibody), signal is detected only from the second padlock B (column 2, TxR channel), and no signal is generated for beta-catenin or for the interaction.

Where both primary antibodies are omitted, no signal is generated from any of the padlocks (bottom row).

### Example 2

### Detection of Beta-Catenin, E-Cadherin, and the Interaction Between the Proteins on FFPE Skin Tissue Slides.

Similarly to Example 1, an experiment was performed using the format of FIG. 6 to detect the proteins beta-catenin (target molecule A) and E-cadherin (target molecule B) in FFPE samples of skin tissue.

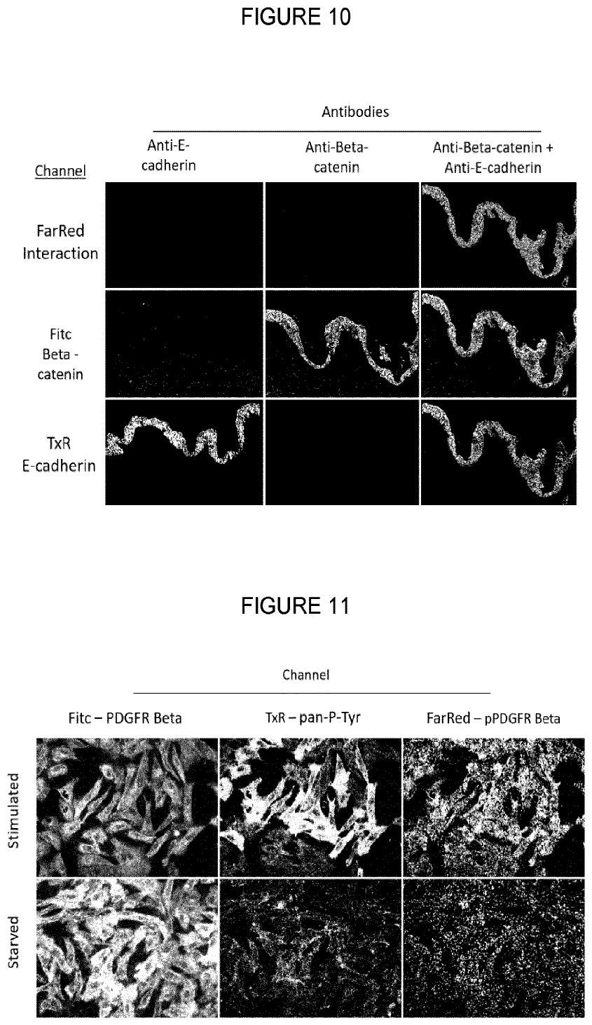

FFPE skin tissue slides were dewaxed with a xylene-based deparaffinization method and rehydrated for 15 min. The slides were then incubated at 125° C. at high pressure in a pressure cooker in citric acid, washed and blocked with a blocking reagent to minimize nonspecific binding. Tissues were then incubated with primary antibodies as described in Example 1 and proximity probe pairs A and B together with the 3 padlock probes were used analogously to detect the individual proteins and their interaction as described in Example 1. Thus, experiments were performed using both primary antibodies, or only one, and the fluorescent signals were read in three channels for the different fluorophores used to detect the individual proteins (FITC for beta-catenin; TxR for E-cadherin and FarRed for the interaction). The results are shown in FIG. 10.

The panel of images in FIG. 10 shows in columns (1) to (3) the signals obtained using the primary antibodies (1) anti-E-cadherin alone, (2) anti-beta-catenin alone, and (3) both anti-E-cadherin and anti-beta-catenin, in the 3 channels for the respective fluorophores, The first row, for the FarRed (interaction) channel, shows that a signal is only obtained when both primary antibodies are present (row 1, column 3); no signal is generated when only one primary antibody is present (row 1, columns 1 and 2). The second row, for the FITC (beta-catenin) channel, show signals when the anti-beta-catenin primary antibody is used alone (column 2) or together with the anti-E-cadherin primary antibody (column 3). The third row, for the TxR (E-cadherin) channel, show signals when the anti-E-cadherin primary antibody is used alone (column 1) or together with the anti-B-catenin primary antibody (column 3). Thus, in rows two and three, the signal is generated in all the conditions where the assay reaction target (as detected by primary antibody) is present, but not when it is not detected (no primary antibody for the target molecule), resulting in a signal in two of the images per row.

### Example 3

Detection of PDGF Receptor-Beta Phosphorylation (pPDGFR-Beta) on BJ-hTert Cells

An experiment was performed using the format of the assay method depicted in FIG. 6 to detect the protein PDGFR beta on BJ-hTert cells, and its phosphorylation (presence of a phosphorylated tyrosine residue) following stimulation of the cells with a PDGFR ligand. Cells starved and not stimulated with a ligand were used as a negative control for the presence of phosphorylation on PDGFR. Primary antibodies directed to PDGFRbeta (target molecule A) and to pan-phospho-tyrosine (target molecule B) were used and detected using proximity probe pairs A and B directed to the primary antibodies for targets A and B respectively, together with padlock probes specific respectively for the nucleic acid domains of the probes. The first probe of pair A was detected by a first padlock probe (A) to detect PDGFRbeta (FITC channel), the first probe of pair B was detected by a second padlock probe (B) to detect phospho-tyrosine Texas Red (TxR) channel). The second probes of pairs A and B were detected by a third padlock probe to detect the pPDGRbeta, namely the phosphorylation of PDGFRbeta (i.e. to detect the presence of the phosphate group on the protein (AB)) (FarRed channel) in ligand-stimulated versus starved cells.

BJ-hTert cells were starved in serum-free growth media overnight and were after that stimulated with PDGFbb for 45 min on ice or left as a control. The cells were then blocked and contacted with one or both primary antibodies, following by assay reactions with the proximity probe pairs A and B and the three padlock probes, to determining the presence of the primary antibodies. As previously, the RCA reaction products of ligated and circularised padlock probes were detected. The results are shown in FIG. 11.

As can be seen from FIG. 11, PDGFR-beta phosphorylation is detected by detecting the presence of both the anti-PDGFR beta antibody and the anti-pan phosphor-tyrosine (pan-P-Tyr) antibody in proximity, i.e. by detecting the interaction (column 3). Detection of anti-PDGFRbeta alone (column 1) or phospho-tyrosine alone (column 2) allows for the analysis of total PDGFR-beta and total pan-P-Tyr in the sample. In the stimulated condition (top row), there is an increased amount of signal for the total phosphorylation and phosphorylated receptor detection, compared to the starved cells (bottom row). The total receptor signal is also slightly downregulated in the stimulated cells.

### Example 4

### Detection of the Proteins Beta-Catenin and E-Cadherin, and the Interaction Between Them, and the Proteins Lamin A and Lamin B and the Interaction Between Them on MCF7 Cells

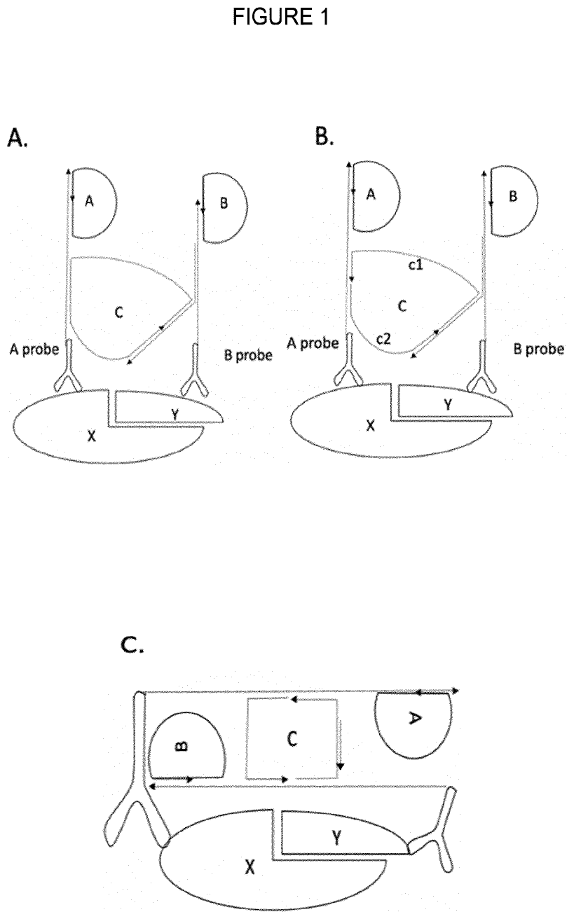

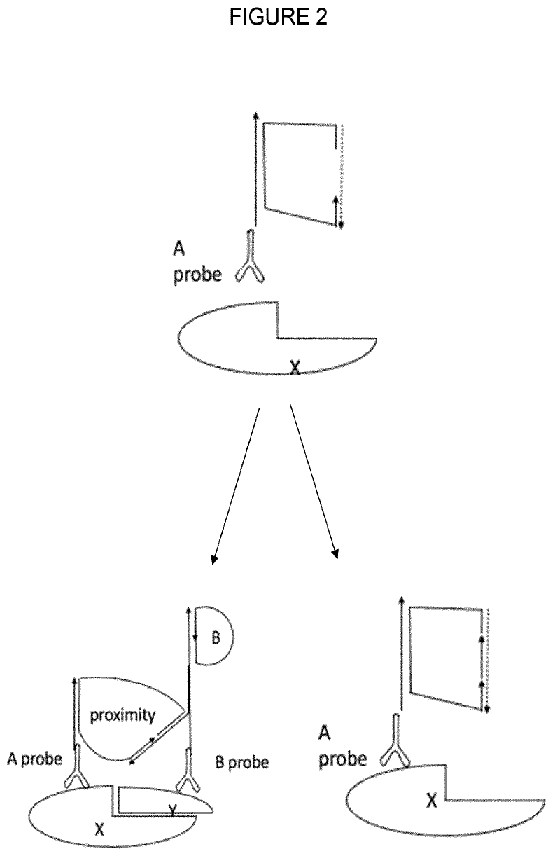

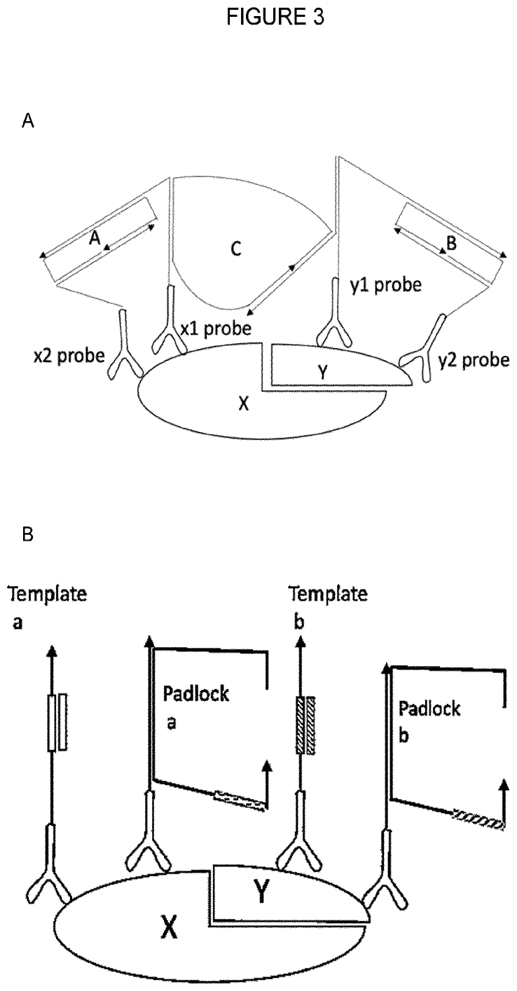

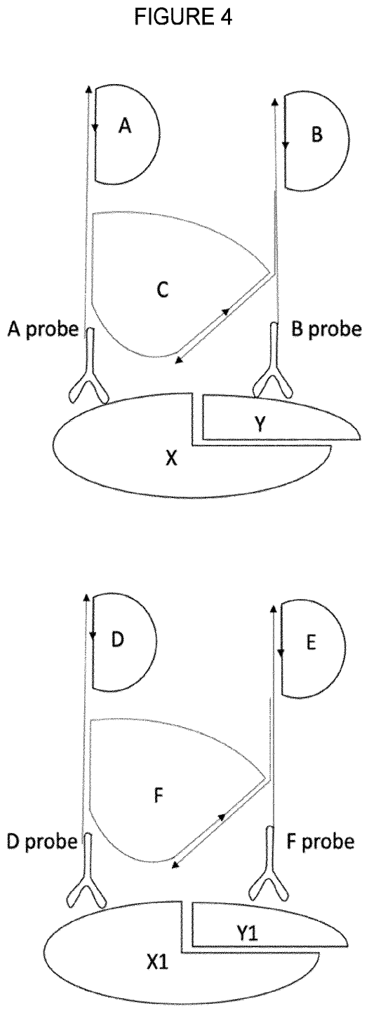

This experiment was performed to demonstrate the detection of proteins and their interaction using a different assay format. In particular this example verifies the assay format as depicted in FIG. 1A.

Two pairs of interacting proteins were selected as targets: beta-catenin (X) and E-cadherin (Y), and lamin A (X) and lamin B1 (Y), which are located on the cell membrane or in the nuclei, respectively. The proteins X and Y individually were detected using probes A and B respectively, together with padlocks A and B, as depicted in FIG. 1A. Probes A and B were used together as a proximity probe pair together with padlock C to detect the interaction between proteins X and Y.

The design of FIG. 1A was modified to incorporate the use of a blocking oligonucleotide hybridized to padlock C. This was done to prevent the premature interaction of the padlock with its ligation template (i.e. with its target) during probe incubation, thus preventing the non-specific signals. The blocking oligonucleotide was displaced by the padlock target (nucleic acid domain of the probe B).

Probe A was prepared by hybridizing padlock A and proximity padlock (padlock C) to the nucleic acid domain of probe A. Probe B was prepared by hybridising (i) padlock B and (ii) the ligation template for padlock C to the nucleic acid domain of probe B to create a partially double-stranded nucleic acid domain for probe B. The padlocks and ligation template for padlock C were hybridized in a ratio of 5:1 to ensure maximum hybridization.

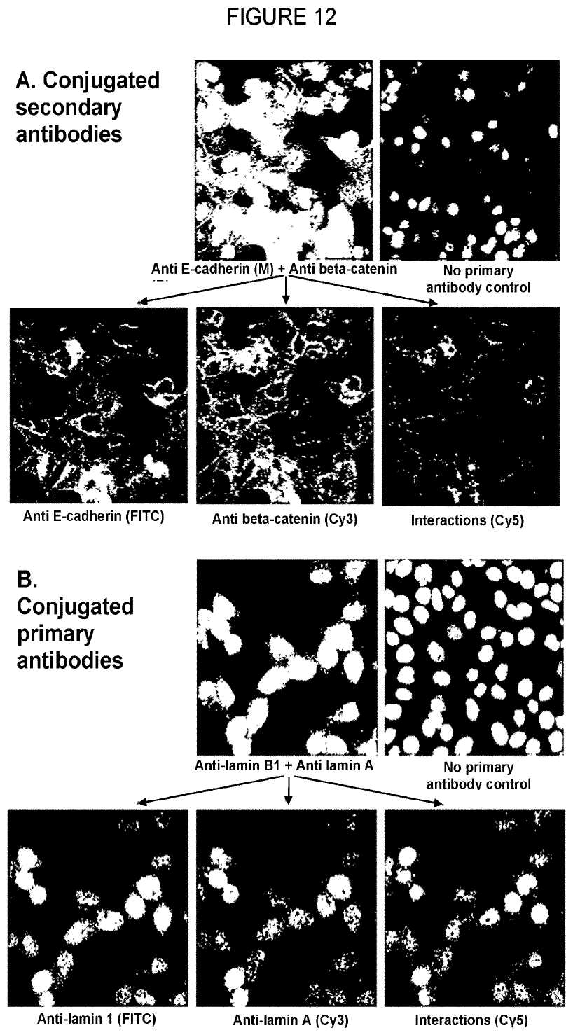

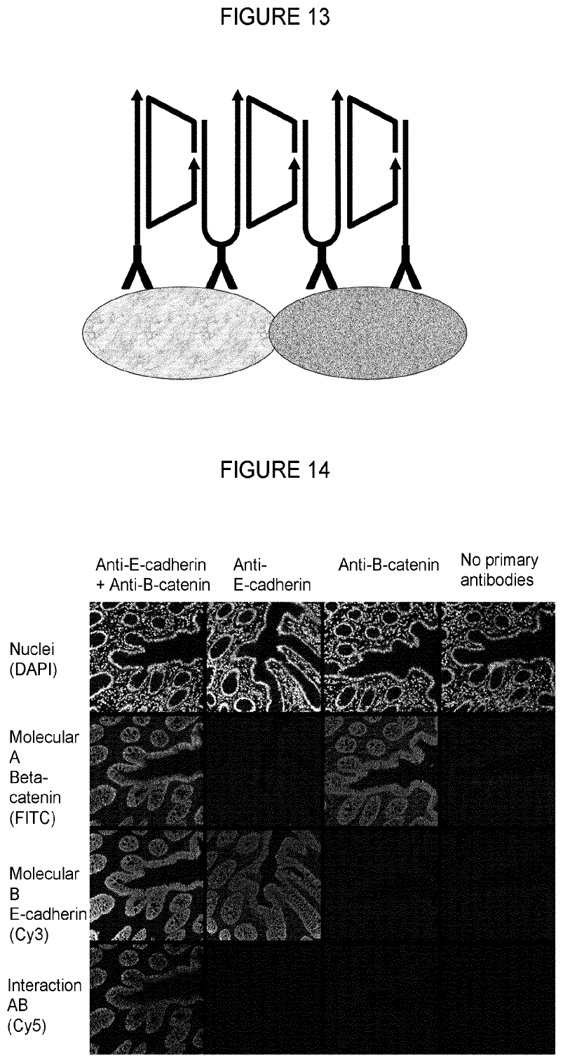

Detection of E-cadherin and beta-catenin was performed using probes A and B as secondary reagents. The MCF7 cell samples were incubated with the Anti E-cadherin (from Mouse) and Anti Beta-catenin (from Rabbit) antibodies targeting E-cadherin and Beta-catenin in cells. Probes A and B probes comprising nucleic acid domains conjugated to secondary antibodies against the primary antibodies together (3 nM each) and hybridized with the padlocks or ligation templates were applied to detect the individual proteins and their interactions. The results are shown in FIG. 12A

Detection of lamin A and lamin B was performed using probes A and B as primary reagents. The primary antibodies Anti Lamin B1 (from Mouse) and Anti Lamin A (from Rabbit), were conjugated with the nucleic acid domains and hybridized with padlocks or ligation templates. 20 nM of both A and B probes were used for detecting Lamin B1, Lamin A, and their interactions. The results are shown in FIG. 12B.

In both cases, after probes A and B have been bound their targets, and excess reagents have been removed by washes, ligation reactions, followed by rolling cycle amplification were initiated. Fluorophore-labelled oligonucleotides complementary to each of the three circularised padlocks were used to detect the RCA products, generating three different signals: two representing the expression of each participating protein and one revealing the interaction level.

### Example 5

### Demonstration of Assay Automation

This example demonstrates the format of the assay method depicted in FIG. 6 and run using an automated slide stainer instrument.

Detection of Beta-catenin (target molecule A), E-cadherin (target molecule B) and their interaction (interaction AB) in human healthy FFPE colon tissue slides was performed on the Leica Bond RX Fully Automated Research Stainer.

The tissue slide was placed in the Leica Bond instrument and deparaffinized using the Bond Dewax Leica protocol. Next, antigen retrieval was performed with the Leica Bond Epitope Retrieval 2 (RE2) for 40 minutes at 100° C.

The proteins Beta-catenin and E-cadherin were detected by incubating the tissue with anti-rabbit and anti-mouse primary antibodies, respectively. Absence of primary antibodies was used as a negative control. Three padlock probes were used to detect Beta-catenin (A), E-cadherin (B) and their interaction (AB). After hybridisation to their respective targets (nucleic acid domains of the proximity probes), the padlocks were circularised by ligation, subjected to RCA to generate RCA products and detected with fluorescently labelled detection probes complimentary to their respective RCA products. The first probe of pair A was detected by a first padlock probe (A) to detect B-catenin (FITC channel), the first probe of pair B was detected by a second padlock probe (B) to detect E-cadherin (Cy3 channel). The second probes of pairs A and B were detected by a third padlock probe to detect the interaction (AB) (Cy5 channel). DAPI staining and slide mounting was down off instrument and before image scanning. The results are shown in FIG. 14.

### Example 6

### Detection of Proteins When no Protein-Protein Interaction is Expected

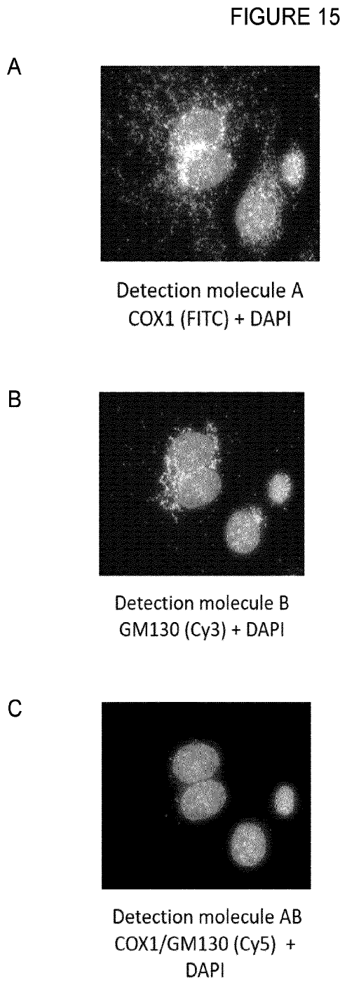

With this example, we demonstrate that an interaction signal is only generated when the interaction partners are in close proximity. FIG. 15 shows the results of using the format depicted in FIG. 6 to detect the mitochondrial protein COX1 (target molecule A; FITC channel) (FIG. 15A) and the golgi protein GM 130 (target molecule B; Cy3 channel) (FIG. 15B) in MCF-7 cells. No interaction signal (AB) is expected as the proteins reside in different subcellular compartments and at a distance that is most likely greater than the limits of the assay. Indeed, no AB interaction signal is observed (Cy5 channel) (FIG. 15C). Each image (FIG. 15 A, B, C) includes a DAPI stain to guide the expected location of the other targets.

1. A method for detecting two target molecules in a sample, and detecting an interaction between the two target molecules, the method comprising:
(i) contacting the sample with (a) a first probe, or a first proximity probe pair, for detection of a first target molecule and (b) a second probe, or a second proximity probe pair for detection of a second target molecule;
wherein said probes each comprise a binding domain capable of binding directly or indirectly to their target molecule and a nucleic acid domain, and said first and second probes, or one of the probes of the first proximity probe pair and one of the probes of the second proximity probe pair, together form a third proximity probe pair for detection of the interaction between the first and second target molecules;
(ii) performing a first assay reaction to detect the nucleic acid domain of the first probe or of at least one member of the first proximity probe pair, thereby detecting the first target molecule;
(iii) performing a second assay reaction to detect the nucleic acid domain of the second probe or of at least one member of the second proximity probe pair, thereby detecting the second target molecule;
(iv) performing a third assay reaction to detect an interaction between the first and second target molecules, wherein said third assay is a proximity assay using the third proximity probe pair, wherein, when said first and second target molecules are present in proximity in an interaction, the nucleic acid domains of the third proximity probe pair interact with each other directly or indirectly to generate a nucleic acid product, and said product is detected to detect the interaction between the first and second target molecules.

2. The method of claim 1, wherein the first and second assays comprise quantifying the amount of target molecule which is detected, and the third assay comprises quantifying the amount of target molecules which are present in the interaction.

3. The method of claim 1 or claim 2, wherein:
(a) said first assay reaction utilises a first nucleic acid reagent capable of hybridising to the nucleic acid domain of the first probe or of at least one member of the first proximity probe pair, and said first reagent or the hybridisation thereof is detected;
(b) said second assay reaction utilises a second nucleic acid reagent capable of hybridising to the nucleic acid of the second probe or of at least one member of the second proximity probe pair, and said second reagent or the hybridisation thereof is detected;
(c) said third assay reaction either (i) utilises a third nucleic acid reagent capable of hybridising to both the nucleic acid domains of the third proximity probe pair to generate the nucleic acid product of the proximity probe interaction, or (iii) utilises one of the first or second nucleic acid reagents, which is capable of hybridising also to the nucleic acid domain of the other member of the third proximity probe pair to generate the nucleic acid product of the third proximity probe pair interaction.

4. The method of any one of claims 1 to 3, wherein the nucleic acid domain of said first probe or of one member of the first proximity probe pair comprises a first tag sequence which is detected to detect the first target molecule; the nucleic acid domain of the second probe or of one member of the second proximity probe pair comprises a second tag sequence which is detected to detect the second target molecule; and the nucleic acid domains of said first and second probes or the members of the first and second proximity pairs which constitute the third proximity probe pair each additionally comprise a separate region of complementarity capable of mediating the interaction between the nucleic acid domains of the third proximity probe pair.

5. The method of claim 4, wherein the first and second tag sequences constitute binding sites for the first and second nucleic acid reagents respectively.

6. The method of any one of claims 3 to 5, wherein in the first assay a first nucleic acid product is generated from or using the first nucleic acid reagent, in the second assay a second nucleic acid product is generated from or using the second nucleic acid reagent, and a third nucleic acid product is generated in the third assay, wherein the first, second and third nucleic acid products are distinguishable from one another by sequence and are detected to detect the first and second target molecules and the interaction between them.

7. The method of claim 6, wherein the first, second and third nucleic acid products are amplified, and the resulting amplicons are detected.

8. The method of any one of claims 3 to 5, wherein an extension product, ligation product, hybridisation product or amplification product of the nucleic acid reagents is generated and detected.

9. The method of claim 1, comprising:
in or after step (i) contacting the probes with at least first and second nucleic acid substrate molecules, wherein the first substrate molecule hybridises to the nucleic acid domain of the first probe or one of the probes of the first proximity probe pair and the second substrate molecule hybridises to the nucleic acid domain of the second probe or one of the probes of the second proximity probe pair;
in step (ii) generating a first nucleic acid product from the first nucleic acid substrate;
in step (iii) generating a second nucleic acid product from the second nucleic acid substrate;
wherein the product of step (iv) is a third nucleic acid product, wherein the first, second and third nucleic acid products are distinguishable from one another by sequence;
in step (v) optionally generating amplification products from said first, second and third nucleic acid products; and
in step (vi), detecting said nucleic acid or amplification products, wherein the first nucleic acid or amplification product indicates the first target molecule, the second nucleic acid or amplification product indicates the second target molecule and the third nucleic acid or amplification product indicates the interaction between the first and second target molecules.

10. The method of claim 9, wherein the relative levels of the first, second and third nucleic acid or amplification products respectively indicate the relative levels of the two target molecules and the proportion of the two target molecules interacting with one another.

11. The method of claim 9 or 10, wherein a third substrate nucleic acid molecule is contacted with the probes, which hybridises with both nucleic acid domains of the third proximity probe pair, and the third nucleic acid product is generated from said third substrate nucleic acid molecule.

12. The method of any one of claims 9 to 11, wherein the third nucleic acid molecule is generated from a first or second substrate molecule upon its interaction with the nucleic acid domains of the proximity probes of the third proximity probe pair.

13. The method of any one of claims 9 to 12, wherein the first and second nucleic acid substrate molecules, and where present the third nucleic acid substrate molecule, are padlock probes provided in one or more parts.

14. The method of any one of claims 9 to 13, wherein the first, second, and third amplification products are RCA products (RCPs).

15. The method of any one of claims 9 to 14, wherein the first, second, and third nucleic acid products are generated by ligation to form circular nucleic acid molecules.

16. The method of any one of claims 9 to 15, wherein the first, second, and third nucleic acid products are generated by direct or indirect ligation of padlock probes, wherein said padlock probes are capable of hybridising to the nucleic acid domains of the probes and said ligations are templated by the nucleic acid domains of the probes.

17. The method of any one of claims 9 to 16, wherein the two target molecules are in complex with one another, or wherein the first target molecule is a protein, and the second target molecule is a post-translationally added modifying group.

18. The method of any one of claims 1 to 11 or 13 to 17, wherein the method comprises:
(i) contacting the sample with:
(a) a first proximity probe, for the first target molecule, wherein the first proximity probe comprises a first nucleic acid domain comprising one or more single-stranded regions, wherein the first nucleic acid domain comprises a first padlock binding site for a first padlock probe and a hybridisation sequence capable of hybridising to a third padlock probe, the first padlock binding site and the hybridisation sequence both being located within the one or more single-stranded regions;
(b) a second proximity probe, for the second target molecule, wherein the second proximity probe comprises a second nucleic acid domain comprising one or more single-stranded regions, wherein the second nucleic acid domain comprises a second padlock binding site for a second padlock probe and a hybridisation sequence capable of hybridising to the third padlock probe, the second padlock binding site and the hybridisation sequence both being located within the one or more single-stranded regions;
(c) a first padlock probe which comprises at its 5′ and 3′ ends target binding sequences capable of hybridising to the first padlock binding site of the first proximity probe;
(d) a second padlock probe which comprises at its 5′ and 3′ ends target binding sequences capable of hybridising to the second padlock binding site of the second proximity probe; and
(e) a third padlock probe which comprises:
(I) a single circularisable oligonucleotide comprising a target-binding sequences at its 5′ and 3′ ends capable of hybridising to the hybridisation sequence of the second proximity probe and in the backbone region between the 5′ and 3′ ends an anchor sequence capable of hybridising to the hybridisation sequence of the first proximity probe, and wherein when applied to the sample the anchor sequence is hybridised to the hybridisation sequence of the first proximity probe; or
(II) two circularisation oligonucleotides together forming a two-part padlock probe, each circularisation oligonucleotide comprising at its 5′ and 3′ ends a first target binding sequence capable of hybridising to the hybridisation sequence of the first proximity probe and a second target binding sequence capable of hybridising to the hybridisation sequence of the second proximity probe, such that when the first and second proximity probes are in proximity each circularisation oligonucleotide hybridises to the nucleic acid domains of both proximity probes, and the respective 5′ and 3′ ends of the two circularisation oligonucleotides are brought into juxtaposition for ligation, directly or indirectly, to each other to form a circle;
(ii) where the 5′ and 3′ ends of the padlock probes have hybridised to their respective binding sites or hybridisation sequences with a gap between them, performing a gap-filling reaction, and ligating the hybridised padlock probes and one or two circularisation oligonucleotides, thereby generating a first circular nucleic acid product from the first padlock probe, a second circular nucleic acid product from the second padlock probe, and a third circular nucleic acid product from the third padlock probe, wherein the first, second and third circular nucleic acid products are distinguishable from one another by sequence.
(iii) amplifying the first, second and third circular nucleic acid products by rolling circle amplification (RCA) to generate a first, second and third RCA product (RCP); and
(iv) detecting the first, second and third RCP, wherein the first RCP indicates the first target molecule, the second RCP indicates the second target molecule, and the third RCP indicates the interaction between the two target molecules.

19. A method as claimed in any one of claims 1 to 11 or 13 to 17, wherein the method comprises:
(i) contacting the sample with:
(a) a first proximity probe pair comprising a first and second proximity probe for the first target molecule, each proximity probe comprising a nucleic acid domain comprising one or more single-stranded regions, wherein the nucleic acid domain of the first proximity probe comprises a first padlock binding site for a first padlock probe and the nucleic acid domain of the second proximity probe comprises a hybridisation sequence capable of hybridising to a third padlock probe, the first padlock binding site and the hybridisation sequence both being located within the one or more single-stranded regions;
(b) a second proximity probe pair comprising a first and second proximity probe for the second target molecule, each proximity probe comprising a nucleic acid domain comprising one or more single-stranded regions, wherein the nucleic acid domain of the first proximity probe comprises a second padlock binding site for a second padlock probe and the nucleic acid domain of the second proximity probe comprises a hybridisation sequence capable of hybridising to the third padlock probe, the second padlock binding site and the hybridisation sequence both being located within the one or more single-stranded regions;
(c) a first padlock probe which comprises at its 5′ and 3′ ends target=binding sequences capable of hybridising to the first padlock binding site of the first proximity probe of the first proximity probe pair;
(d) a second padlock probe which comprises at its 5′ and 3′ ends target-binding sequences capable of hybridising to the second padlock site of the first proximity probe of the second proximity probe pair;
(e) a third padlock probe which comprises:
(I) a single circularisable oligonucleotide comprising target-binding sequences at its 5′ and 3′ ends capable of hybridising to the hybridisation sequence of the second proximity probe of the second proximity probe pair, and an anchor sequence capable of hybridising to the hybridisation sequence of the second proximity probe of the first proximity probe pair, and wherein when applied to the sample the anchor sequence is hybridised to the hybridisation sequence of the second proximity probe of the first proximity probe pair; or
(II) two circularisation oligonucleotides together forming a two-part padlock probe, each circularisation oligonucleotide comprising at its 5′ and 3′ ends a first target-binding sequence capable of hybridising to the hybridisation sequence of the second proximity probe of the first proximity probe pair and a second target-binding sequence capable of hybridising to the hybridisation sequence of the second proximity probe of the second proximity probe pair, such that when the first and second proximity probes are in proximity, each circularisation oligonucleotide hybridises to the nucleic acid domains of both second proximity probes, and the respective 5′ and 3′ ends of the two circularisation oligonucleotides are brought into juxtaposition for ligation, directly or indirectly, to each other to form a circle;
(ii) where the 5′ and 3′ ends of the padlock probes have hybridised to their respective binding sites or hybridisation sequences with a gap between them, performing a gap-filling reaction, and ligating the hybridised padlock probes, thereby generating a first circular nucleic acid product from the first padlock probe, a second circular nucleic acid product from the second padlock probe, and a third circular nucleic acid product from the third padlock probe, wherein the first, second and third circular nucleic acid products are distinguishable from one another by sequence;
(iii) amplifying the first, second and third circular nucleic acid products by rolling circle amplification (RCA) to generate a first, second and third RCA product (RCP); and
(iv) detecting the first, second and third RCP, wherein the first RCP indicates the first target molecule, the second RCP indicates the second target molecule, and the third RCP indicates the interaction between the two target molecules.

20. A method as claimed in any one of claims 1 to 3, 6 to 10, or 12 to 17, wherein the method comprises:
(i) contacting the sample with:
(a) a first proximity probe for the first target molecule, wherein the first proximity probe comprises nucleic acid domain (e.g. a single-stranded nucleic acid domain) hybridised to a first padlock probe, wherein the 5′ and 3′ ends of the first padlock probe are hybridised to a blocking oligonucleotide;
(b) a second proximity probe for the second target molecule, wherein the second proximity probe comprises a nucleic acid domain comprising one or more single-stranded regions, and wherein the nucleic acid domain comprises a first padlock binding site capable of hybridising to the 5′ and 3′ ends of the first padlock probe, and a second padlock binding site for a second padlock probe, the padlock binding sites both being located within the one or more single-stranded regions; and
(c) a second padlock probe, which comprises at its 5′ and 3′ ends target-binding sequences capable of hybridising to the second padlock binding site of the second proximity probe;
such that when the first and second proximity probe are in proximity the blocking oligonucleotide is displaced from the first padlock probe by the single-stranded region comprising the first padlock binding site of the second proximity probe,
wherein the blocking oligonucleotide and/or the first padlock binding site of the second proximity probe comprise a gap sequence located between complementary binding sites capable of hybridising to the 5′ and 3′ ends of the first padlock probe, such that the hybridised 3′ and 5′ ends of the first padlock probe are separated by a gap;
(ii) when the 5′ and 3′ ends of the padlock probes have hybridised to their respective binding sites with a gap between them, performing a gap-filling reaction, and ligating the hybridised padlock probes, thereby generating a first circular nucleic acid product from the first padlock probe hybridised to the blocking oligonucleotide, a second circular nucleic acid product from the second padlock probe, and a third circular nucleic acid product from the first padlock probe hybridised to the first padlock binding site of the second proximity probe, wherein the first, second and third circular nucleic acid products are distinguishable from one another by sequence;
(iii) amplifying the first, second and third circular nucleic acid products by rolling circle amplification (RCA) to generate a first, second and third RCA product (RCP); and
(iv) detecting the first, second and third RCP, wherein the first RCP indicates the first target molecule not interacting with the second target molecule, the second RCP indicates the second target molecule, and the third RCP indicates the interaction between the two target molecules.

21. A method as claimed in any one of claims 1 to 3, 6 to 10, or 12 to 17, wherein the method comprises:
(i) contacting the sample with:
(a) a first proximity probe pair comprising a first and second proximity probe for the first target molecule, wherein the first proximity probe comprises a nucleic acid domain comprising a first padlock binding site, and the second proximity probe comprises a nucleic acid domain hybridised to a first padlock probe which comprises at its 5′ and 3′ ends binding sequences capable of hybridising to the first padlock binding site; and
(b) a second proximity probe pair comprising a first and second proximity probe for the second target molecule, wherein the first proximity probe comprises a nucleic acid domain comprising a second padlock binding site, and the second proximity probe comprises a nucleic acid domain hybridised to a second padlock probe which comprises at its 5′ and 3′ ends binding sequences capable of hybridising to the second padlock binding site;
wherein the binding sequences of the first padlock probe are also capable of hybridising to the second padlock binding site, and/or the binding sequences of the second padlock probe are also capable of hybridising to the first padlock binding site;
wherein the first and second padlock probes each comprise an identifier sequence, and the first and/or second padlock binding sites comprise a gap sequence located between complementary binding sites capable of hybridising to the 5′ and 3′ ends of the respective padlock probes;
(ii) where the 5′ and 3′ ends of a padlock probe have hybridised to their respective binding sites with a gap between them, performing a gap-filling reaction, and ligating the hybridised padlock probes, thereby generating a first circular nucleic acid product from the first padlock probe hybridised to the first padlock binding site, a second circular nucleic acid product from the second padlock probe hybridised to the second padlock binding site, and a third and, optionally, fourth circular nucleic acid product from the first padlock probe hybridised to the second padlock binding site and/or the second padlock probe hybridised to the first padlock binding site, wherein the first, second, third and optionally fourth circular nucleic acid products are distinguishable from one another by sequence;
(iii) amplifying the first, second, third and optional fourth circular nucleic acid products by rolling circle amplification (RCA) to generate a first, second, third and, optionally, fourth, RCA product (RCP); and
(iv) detecting the first, second, third and optional fourth RCP, wherein the first RCP indicates the first target molecule, the second RCP indicates the second target molecule, and the third RCP, and optional fourth RCP, indicate the interaction between the two target molecules.

22. A kit for performing the method of any one of claims 1 to 21, the kit comprising:
(i) a first probe or proximity probe pair for detection of a first target molecule, and a second probe or proximity probe pair for detection of a second target molecule, wherein said probes each comprise a binding domain capable of hybridising directly or indirectly to their target molecule and a nucleic acid domain, and
wherein the first and second probe, or one of the probes of the first proximity probe pair and one of the probes of the second proximity probe pair, together form a third proximity probe pair for detection of the interaction between the first and second target molecules; and
(ii) first and second nucleic acid nucleic acid reagents, wherein the first reagent is capable of hybridising to the nucleic acid domain of the first probe or one of the probes of the first proximity probe pair and the second reagent is capable of hybridising to the nucleic acid domain of the second probe or one of the probes of the second proximity probe pair;
optionally wherein the first and/or second reagent is further capable of hybridising to the nucleic acid domain of the other member of the third proximity probe pair.

23. The kit of claim 22, wherein the first and second nucleic acid reagents are substrate molecules capable of giving rise to distinguishable first and second nucleic acid product.

24. The kit of claim 22 or claim 23, further comprising a third nucleic acid substrate molecule capable of hybridising to both the nucleic acid domains of the third proximity probe pair 25. The kit of any one of claims 22 to 24, wherein the first and second nucleic acid substrate molecules, and where present the third nucleic acid substrate molecule, are padlock probes provided in one or more parts.

26. The kit of any one of claims 22 to 25, wherein:
(i) the binding domain of at least the first probe, or the binding domains of at least the first proximity probe pair, is an antibody or antigen-binding fragment thereof; and/or
(ii) the kit further comprises a ligase; and/or.
(iii) the kit further comprises a strand-displacing nucleic acid polymerase, preferably phi29 polymerase.
